(19) United States
(12) Patent Application Publication
(10) Pub. No.: US 2014/0127179 A1
(43) Pub. Date: May 8, 2014

(54) Natural Killer Cell Formulations

(71) Applicant: Jerry Thomas THORNTHWAITE, Henderson, TN (US)

(72) Inventor: Jerry Thomas THORNTHWAITE, Henderson, TN (US)

(73) Assignee: SCIENTIFIC FORMULATIONS, LLC, Chester, VA (US)

(21) Appl. No.: 14/070,900

(22) Filed: Nov. 4, 2013

Related U.S. Application Data
(60) Provisional application No. 61/721,649, filed on Nov. 2, 2012.

(51) Int. Cl. A61K 31/716; A61K 31/375; A61K 31/05; A61K 31/198; A61K 31/385; A61K 31/714; A61K 31/355; A61K 33/04; A61K 36/8962; A61K 36/185; A61K 31/12; A61K 33/30

(52) U.S. Cl. CPC A61K 31/716; A61K 31/12; A61K 31/375; A61K 31/05; A61K 31/198; A61K 33/30; A61K 31/714; A61K 31/355; A61K 33/04; A61K 36/8962; A61K 36/185; A61K 31/385
USPC 424/94.1; 514/54; 514/474; 424/641; 514/52; 424/702; 424/754

(57) ABSTRACT

A formulation of supplement is able to boost the natural cells, both increasing the population of the natural cells and enhancing the natural cell's activity. The formulation can be administered via different forms, such as tablet, injection, skin absorption and etc. This formulation comprises 16 different components, including Alpha Lipoic Acid, Arabinoxylin, Curcumin, Garlic, Genistein, Ginseng, Lentinan, Mistletoe, N-Acetylcysteine, Resveratrol, Selenium, Vitamin B Complex, Vitamin C, Vitamin D3, Vitamin E and Zinc. By virtue of enhancing the natural killer cells (NKC) in the body, the formation is helpful for the prevention and treatment of a number of different human or animal diseases, such as cancers and viral infections. In addition, certain synergistic effects have been observed between different components in the formulation, which further enhances the beneficial effect of the formulation.

The current application claims a priority to the U.S. Provisional Patent Application Ser. No. 61/721,649, filed on Nov. 2, 2012.

### FIELD OF THE INVENTION

The present invention relates generally to the formulation of health supplement. More specifically, the present invention is a formulation of supplements to boost the Natural Killer cells (NKC) activity, so as to help to prevent and treat certain human and animal diseases.

### BACKGROUND OF THE INVENTION

Natural Killer cells (NKC) are a group of lymphocytes that is critical for the immune responses of human and animals. It functions mainly as a type of cytotoxic lymphocyte in the innate immune system. More and more studies have found that NKCNKC play a critical role in the immune system with a unique function that are differentiable from that of the common B cells and T cells. NKC function particularly important in body's rapid response to viral infection and tumor formation. It can act as quick as just 3 days after the infection.

NKC do not express T-cell antigen receptors (TCR), T marker CD3 or surface immunoglobulins (Ig) B cell receptors. Rather they usually express the surface markers CD16 (FcγRIII) and CD56. In addition, a majority of human NKC also express CD8.

NKC function in a unique way. Usually, the typically immune cells detect MHC presented on infected cell surfaces, triggering cytokine release, causing lysis or apoptosis. Nevertheless, the NKC are capable of recognizing the stressed cells in the absence of antibodies and MHC, allowing for a much faster immune reaction. In immunology, the NKC belong to the family of large granular lymphocytes (LGL) and constitute the third kind of cells differentiated from the common lymphoid progenitor generating B and T lymphocytes. In the human body, NKC may be derived and/or mature at various locations, such as bone marrow, lymph node, spleen, tonsils and thymus. And they then enter into the circulation. NKC are unique in the immune system as they are able to function in both innate and adaptive immune responses. NKC destroy their targets by releasing substances that damage the offending cell, leading to an immune response that eliminates the cell.

NKC have the capability to recognize and induce the killing of certain infected cells, cancer cells, other types of stressed cells. In addition, they produce a variety of cytokines, including pro-inflammatory cytokines, chemokines, colony-stimulating factors, and other cytokines that function as regulators of body defenses. Concerning the activation and inactivation of themselves, NKC use a dual receptor system in determining whether to kill or not kill human cells to which they bind. These dual receptors are termed the killer activating receptor (KAR) and the killer inhibitory receptor (KIR).

In particular, when cells are under stress, which includes the situation of being infected by microbes such as virus, and developing into tumors, various stress-induced molecules, such as heat shock proteins, extracellular matrix fragments, and altered membrane phospholipids, are produced and presented on the surface of those cells. To start the killing process, KAR on the surface of the NK cell is able to recognize the various stress-induced molecules on the target cell's surface and binding to these molecules. This will send a positive signal that enables the NK cell to induce the death of the cell to which it has bound unless a KIR cancels that signal.

One of the most valuable therapeutic potentials of NKC is their critical functions in the control and treatment or tumor cell development. As an important player of the innate immune system, NKC are able to act almost immediately when certain event occurs, with no need for a prior exposure to the particular antigen or pathogen. In the context of tumor cell development, as cytolytic effector lymphocytes, NKC are able to directly induce the death of tumor cells. More importantly, NKC could conduct it with the absence of cell surface adhesion molecules and/or antigenic peptides. Other important players in the immune system, such as the T cells, are unable to recognize pathogens in the absence of surface antigens. When tumor cells have been detected, NKC will be activated and certain cytokines will be released from them. In addition to functioning to kill the tumor cells, the NKC are also able to trigger other immune responses, such as T cell response. In many cases, the tumor cells may not cause inflammation; and thus they do not trigger the T cell response. Yet after activation, NKC are able to release a number of different cytokines, such as TNFα, IL-10 and etc. The release of these cytokines will induce macrophages, dendritic cells, and neutrophils and etc. And these immune cells will further induce the antigen-specific T cell and B cell responses.

In view of the foregoing, the NKC have huge potential in the treatment of cancers and viral infections. Yet one drawback for employing NKC for the treatment of these diseases is the limited number of NKC in the blood, as well as the various activities of the NKC; that being said, some NKC have lower activities due to the lack of certain important receptors or factors, such as the 58 V/V allele and CD8. In light of this issue, one possible approach to achieve the ultimate treatments of the cancer or viral infection would be to boost the activity of the human NKC, which could be accomplished by either increasing the population of the NKC or enhancing NKC′ activity, or both.

Therefore, one objective of the present invention is via immune system stimulation to increase the number and vitality of the NKC. It is understandable that if a large enough number of NKC are activated to a level required by a potent anti-malignancy action, they could be able to destroy most of the cancer cells in the human or animal body, thus providing a promising way to cure the cancer or viral infective diseases. This has been supported by most of the studies in the art and most of the immunology books as well.

On the other hand, there could be various ways towards the objective of boosting the NKC in the body, e.g., by medical drugs to boost the NKC, through genetically modification or bioengineering to boost the NKC, and so on. However, there is also a natural way to boost the NK cell; it is to say, boost NKC by virtue of certain natural products or supplements. Certain natural food and products, such as herbs, spice, foods, have been found to enhance NK cell activity. However, there is no such a systematic and in depth investigation for the influence of each of them to NKC; and there is no exploration about their synergistic effects in one formulation with the purpose of boosting the human or animal NKC.

It is therefore an objective of the present invention to introduce a formulation of supplements to enhance NK cell in human or animal, and thereby helps to treat human and animal diseases, such as cancer and viral infection.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows two B cells releasing IgM against SRBCs in the presence of complement. Lysed SRBC appear as deflated balloons.

FIG. 2 shows the Natural Killer Cell (CIPFC) completely destroying the surrounding SRBC.

FIG. 3 shows the BSA continuous isotonic gradient enrichment showing four NKC clear plaques, where the SRBC were completely destroyed; staining with Benzidine and Giemsa dyes.

FIG. 4 shows Nomarski optics visualization of the “blebbing effect” of the NKC damage to the P815 cancer cell membranes. The NKC were derived from lymph nodes of mice bearing the P815 cancer cells.

FIG. 5 shows SEM normal villous surface of the P815 cancer cell (FIG. 5a) and the killing of the P815 cancer cell by the injection of the NK cell toxin with resulting “blebbing” of the cancer cell membrane (FIG. 5b).

FIG. 6 shows TEM sequence of events showing a presumptive NK cell approaching the cancer cell (FIG. 6a) and the killing of the cancer cell by the injection of the toxin with resulting “blebbing” of the cancer cell membrane (FIG. 6b).

### DETAIL DESCRIPTIONS OF THE INVENTION

All illustrations of the drawings are for the purpose of describing selected versions of the present invention and are not intended to limit the scope of the present invention.

One objective of the present invention is to develop a particular formulation that is able to boost the NKC and thereby helping to treat human and/or animal diseases, such as cancer and viral infection. Moreover, in order to take advantage of the natural products, this formulation comprises certain natural medicinal substances and supplements. In this way, this formulation of supplements will contribute to the human health and well-being.

NKC play an important role in the quick response to destruct tumor cells and/or virally infected cells within minutes after cell contact, without causing damage to themselves or the surrounding cells. The death of the target cell occurs after the release of cytoplasmic granules from the effector cell, which would be the NKC. These granules contain the pore-forming protein perforin and serine proteases (granzymes). One mechanism of killing involves exocytosis of granules containing serine proteases and perforin.

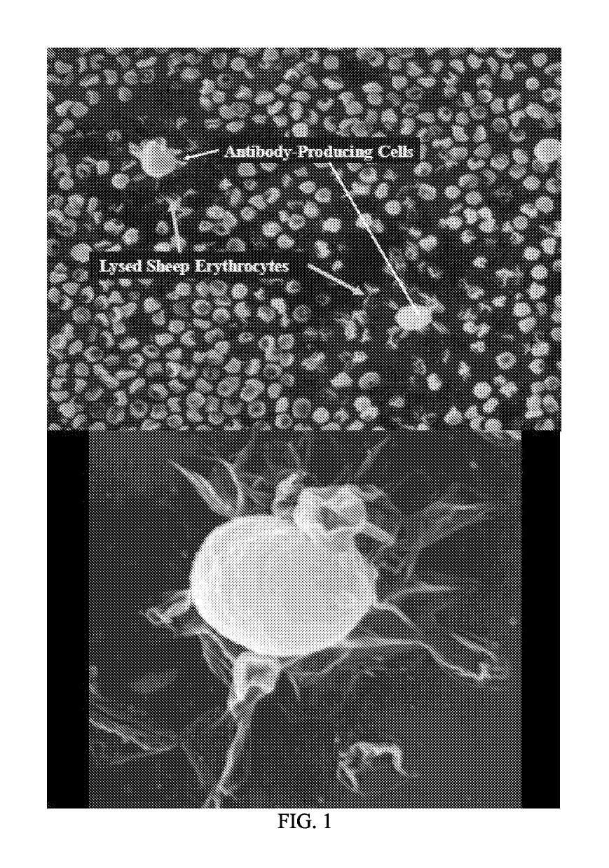

While conducting his thesis research under Dr. Robert Leif from Florida State University in late 1971 at the Papanicolaou Cancer Research Institute in Miami, Dr. Jerry Thornthwaite's thesis project was to transform the Agar-based Jerne Plaque Assay into a non-agar setting assay. This immunological technique was used to detect murine B cells producing IgM against Sheep Red Blood Cells (SRBC), which were derived from the spleen of mice immunized four days earlier with SRBC. This method, termed the Plaque Cytogram Assay (PCA) is able to create cell monolayers on microscope slides through centrifugation in a centrifuge bucket particularly designed to form a glass slide bottom, whereby monolayers of cells distribute themselves onto a slide surface via the centrifugation. This technique also enabled the carefully extraction of supernatants, while subsequently being able to carefully layer solutions such as guinea pig complement and Glutaraldehyde without disturbing the cell monolayer. After the cells were fixed to the slides, the samples could be processed for Light, Scanning Electron Microscopy (SEM) and Transmission Electronic Microscopy (TEM) as presented in the Figures. In reference to FIG. 1, it shows by SEM two antibody secreting B cells forming plaques formed due to the radial release of IgM antibodies in the presence of guinea pig complement. It was at the temperature of 37° C., and for 30 minutes.

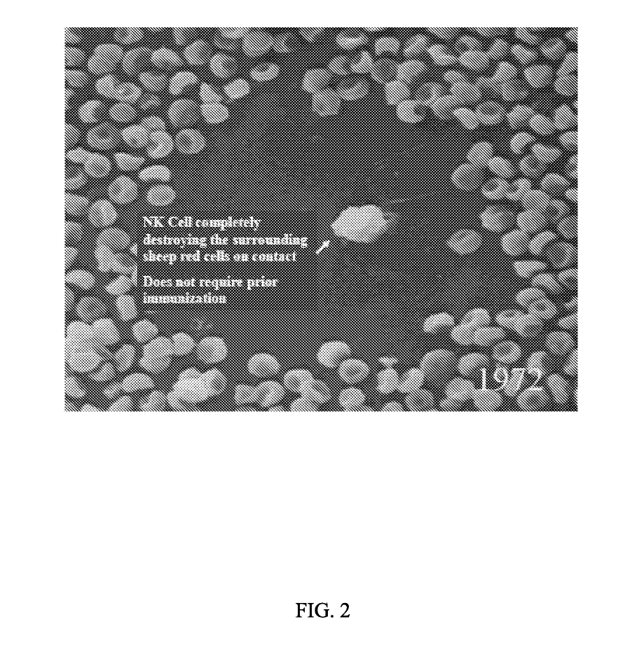

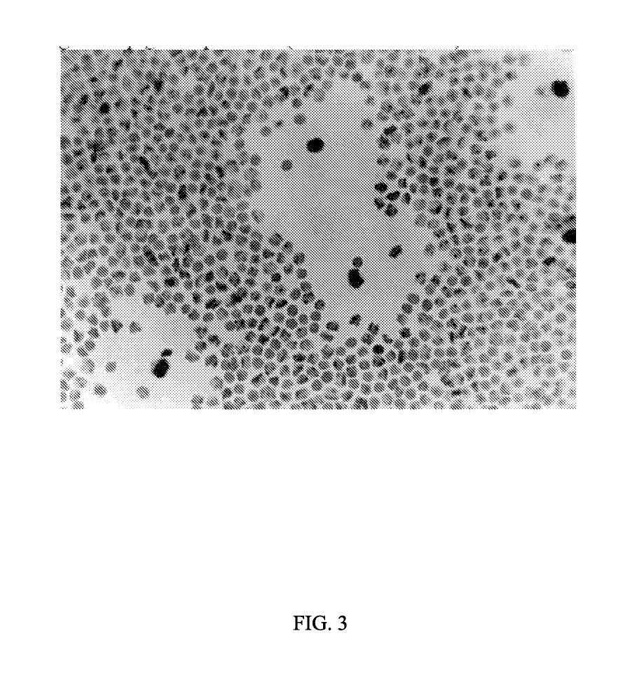

In reference to FIG. 1, it shows two B-cell releasing IgM against SRBCs in the presence of the complement. The lysed SRBC appeared as deflated balloons. In reference to FIG. 2, it shows the Natural Killer Cell (CIPFC) completely destroying the surrounding SRBC. In reference to FIG. 3, it shows the BSA continuous isotonic gradient enrichment showing four NK cell clear plaques, where the SRBC were completely destroyed. The results were stained with Benzidine and Giemsa dyes.

The Membrane Attack Complex action by the NKC results in the deflated balloon appearance of the IgM-complement lysed SRBC. These lysed SRBC are also negative for Hemoglobulin as determined by Benzidine staining.

Since a SRBC monolayer was being produced on the microscope slide surface, immune cellular interactions could be visualized with great accuracy. One of the unusual plaques that were being formed in these immunized mice has been shown in FIG. 2, where the lysed SRBC were not seen in the plaques. In fact, the plaques appeared “clear” under the SEM (FIG. 2) and the light microscopy (FIG. 3), where the central cell was releasing a lytic agent that completely destroyed the surrounding SRBCs in a radial fashion. These clear plaques had been seen regardless if the complement was present or not, thus receiving the initial name Complement Independent Plaque Forming Cells (CIPFC). These CIPFC were seen even in the spleens and lymph nodes of non-immunized mice. The CIPFC were enumerated as being about 10-15% of the lymphoid population in non-immunized mice and were enriched using Bovine Serum Albumin (BSA) continuous isotonic gradients (FIG. 3). Later in 1975, the CIPFC was called as Natural Killer Cells (NKC).

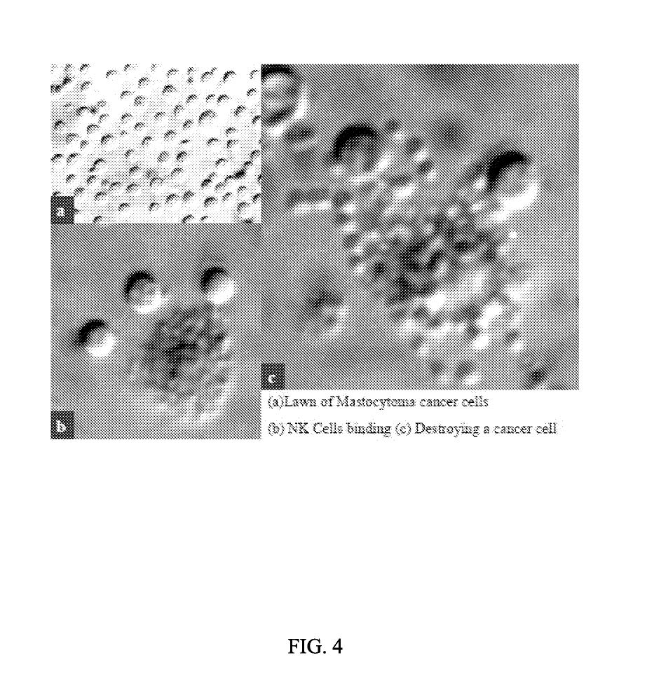

In order to visualize the NKC destroying cancer cells, DBA/2 mice were peritoneally injected with P815 mastocytoma cells, and lymph nodes were derived from these DBA/2 mice. A monolayer of cancer cells were centrifuged onto restricted areas of a microscope slide with a dilute mixture of syngeneic lymph nodes cells using the PCA and then incubated at 37° C. for 30 minutes. In order to document the NK cell's activity of killing the cancer cells, a method had been developed for detecting the killed cells with the Alcian Blue dye. Alcian Blue stains dead cells that were fixed prior to glutaraldehyde fixation; but does not stain cells that were viable before the glutaraldehyde fixation. FIG. 4 shows the results of the incubation of NKC with the target P815 cancer cells. The monolayer of cancer cells was uniform (FIG. 4a); and the potential NKC were seen binding to the dead cancer cells (FIG. 4b). However, the most direct proof was seen when the killing occurred, which caused a “blebbing” of the target cancer cell membranes as the result of the NK cell killing (FIG. 4c).

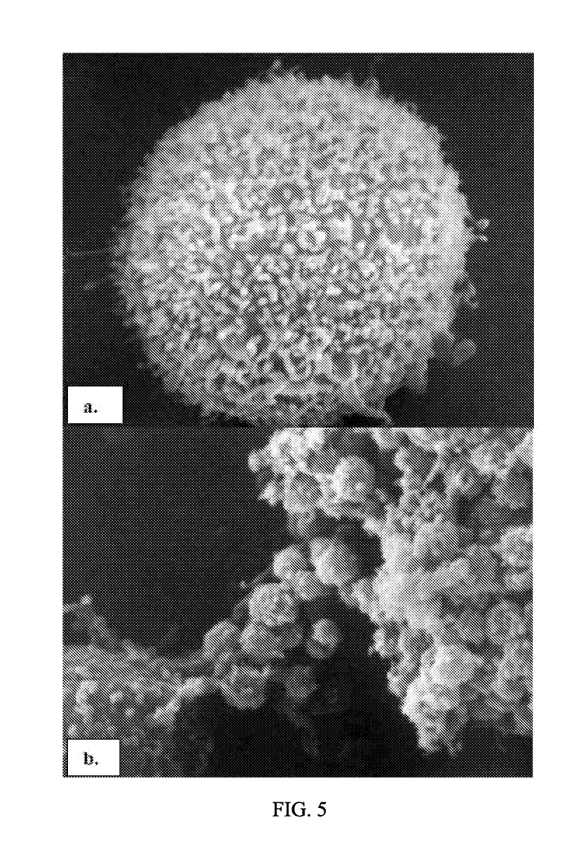

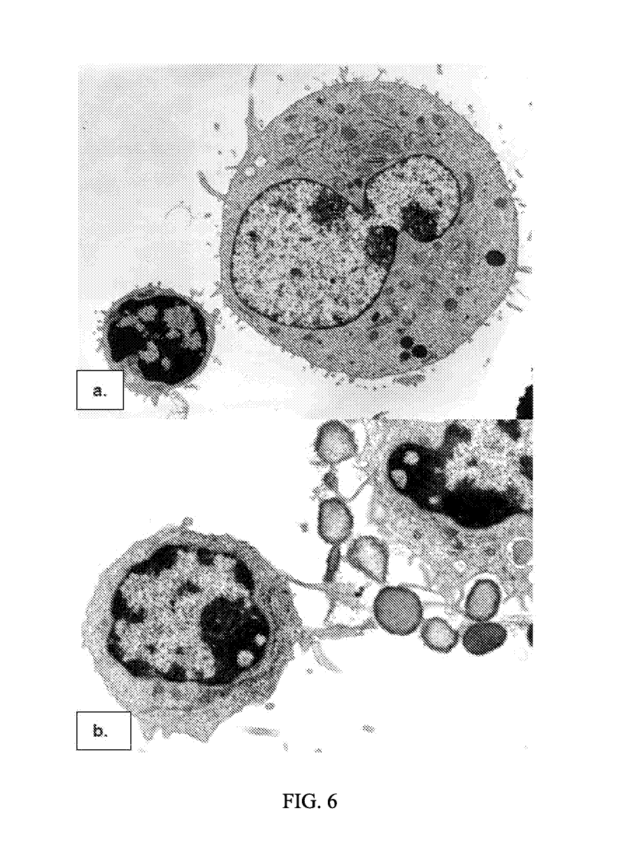

In order to obtain the smallest possible en face section that included only the NK cell activity, Alcian Blue staining revealed as an area of reference where the killing was taking place. FIG. 5 shows an example of the P815 villous surface (FIG. 5a) and the “blebbing” destruction of the P815 cancer cells by SEM (FIG. 5b). Under the TEM, a typical P815 cancer cell is shown in FIG. 6a, while FIG. 6b shows the “fangs” from the NKC injecting the lymphotoxin into the P815 cancer cell with subsequent “blebbing” destruction of the cancer cell.

The term “Natural” Killer Cell was first used by Kiessling and Wigzell in a two part publications in 1975. Later, the term was used routinely in later publications. Publications in 1975 are generally given credit for the NK cell's ability to kill tumor cells as reviewed, for example by Herbeman and Holden in 1978, Herberman and Ortaldo in 1981 and Heberman in 2002. Ultrastrucural studies of supposed NKC were attempted in 1979. During these times, all of the functional evidence for any “natural” activity was (and still is) measured by the ability of unimmunized lymphocytes to kill radioactively labeled tumor cells and release 51Cr, which can have high background counts, making the standardization of the background versus the effects of non-immunized lymphocytes (NK cell activity) difficult to measure.

Since the discovery of the NK cell, as observed by Nomarski optics, SEM and TEM, and enumerated and enriched using BSA continuous gradients, we have been interested in using these NKC to effectively kill cancer cells. In addition to introduce the early studies with NKC, one main objective of the present invention is to develop a formulation that comprises the natural supplements, which is able to boost the NK cell in human and animal, thereby help to treat various diseases, such as cancer and viral infection. In the presentation, at least 16 different supplement components have been identified to enhance the NKC. This study is based on the respective component's influences to the immune modulator activity. Our previous experience with the pharmacological development of the Formulation will allow us to develop a “NKC tablet” of components that may act synergistically to enhance the NK cell activity as a preventative and treatment of existing cancer conditions.

Alpha-Lipoic Acid (ALA)

Abnormal regulation of the inflammatory response is an important component of many diseases such as diabetes, Alzheimer's disease and multiple sclerosis (MS). Alpha Lipoic acid (ALA), naturally produced in the body and can also be found in the food, such as red meat, has been shown to have antioxidant and anti-inflammatory properties and is being pursued as a therapy for these diseases. Oral administration of ALA in vivo results in significant increases in cAMP concentration. The anti-inflammatory effects of ALA are mediated in part by the cAMP/PKA signaling cascade. These novel findings enhance the understanding of the mechanisms of action of ALA. Suppressed interleukin (IL)-12 and IL-18 induced IFNgamma secretion and cytotoxicity in NKC. They hence have demonstrated that ALA may actual inhibit NK cell function via the cAMP signaling pathway. So, the importance of ALA in T cell migration and NK cell activity has been shown.

The anti-oxidant ALA potently suppresses clinical and pathologic disease in the animal model of multiple sclerosis, experimental autoimmune encephalomyelitis, by inhibiting the migration of pathogenic T cells to the spinal cord. Filatova, et al has showed increase sensitivity with two antioxidants, ALA and NAC against 3T3-SV40 fibroblasts with increased lytic activity of NKC.

### Arabinoxylan

Population studies have shown that greater red and processed meat consumption increases colorectal cancer risk, whereas dietary fiber is protective. The non-cellulosic polysaccharides present in cereals (2-8%) are mostly Arabinoxylans, (1→3), (1→4)-beta-glucans, pectins and arabinogalactans. The arabinoxylans are known to absorb large amounts of water and influence significantly the water balance and rheological properties of dough. Isolation, purification, characterization, structural elucidation, functional, and nutritional attributes of cereal heteroxylans particularly the enzymatically modified Arabinoxylin from rice bran. Arabinoxylan rice bran (Biobran) is a potent biological response modifier (BRM) that activates NKC. Biobran stimulated dendritic cells (DCs) induced CD4+ T cell proliferation and their production of cytokines, IFN-gamma, IL-10, IL-17. Results suggested that Biobran functions as a natural adjuvant for DC activation and thus may be used in DC-based vaccine strategies against infections and cancer. The synergistic apoptotic potential of Arabinoxylan rice bran (MGN-3/Biobran) and Curcumin (turmeric) on a human multiple myeloma (MM) cell line U266 has been shown and may establish the foundation for in vivo studies that could have therapeutic implications. Biobran enhances the effects of interventional therapies for the treatment of hepatocellular carcinoma as shown in a three-year randomized clinical trial. The antitumor activity of wheat bran Arabinoxylans may also be mediated via the improvement in the immune response. Arabinoxylan-oligosaccharides reduce preneoplastic lesions in the colon of rats that were treated with 1,2-dimethylhydrazine and suggest a chemopreventive effect on colon carcinogenesis that should be confirmed in a long-term carcinogenesis experiment. BioBran was tested for its possible effects on in vitro maturation of human dendritic cells (DC). It is a potent enhancer of DC maturation and suggests that BioBran might be useful to create the environment that favors DC maturation. Biobran induced oncostatic activity by modulating lipid peroxidation, augmenting the antioxidant defense system and protecting against oxidative stress. Data also showed that non-tumor-bearing mice intramuscularly injected with Biobran resulted in a twofold increase in NKC activity showing clinical implications for the treatment of solid cancers. Gollapudi and Ghoneum demonstrated that Biobran is an effective chemo-sensitizer and may represent a potential novel adjuvant for the treatment of breast cancer. Both wheat bran Arabinoxylans and gut flora-mediated fermentation products protect human colon cells from genotoxic activities of 4-hydroxynonenal and hydrogen peroxide. Breast cancer cell lines undergo apoptosis following phagocytosis of S. cerevisiae. Biobran has been shown to enhance this effect. Hydrolyzed water-soluble corn husk Arabinoxylan has the ability to increase the level of immunopotentiating activity without causing over response of immunological reaction even if it is administrated orally to mice. Hydrolyzed Rice Bran was shown to be useful in reducing the physical stress associated with acute respiratory tract infection. Biobran enhances murine NK cell activity of aged mice and may be useful for enhancing NK cell function in aged humans. Biobran is a potent activator of human NK cell function in vivo and in vitro.

### Curcumin

Based on basic science research and suggestive ecologic level data on cancer incidence and spice consumption, improving epidemiologic assessment of this potentially chemo-preventive food item derived from the spice, Curry, may enhance the understanding of diet and cancer risk. Urothelial carcinoma has the highest lifetime treatment cost of any cancer, making it an ideal target for preventative therapies. There is increasing evidence to suggest that many degenerative or pathological processes, such as aging, cancer, and coronary heart disease, are related to reactive oxygen species and radical-mediated reactions. Inflammation plays a major role in the development of various neurodegenerative diseases, including Alzheimer's disease, Parkinson's disease, multiple sclerosis, brain tumor, and meningitis, the incidence of certain neurodegenerative diseases among people living in the Asian subcontinent, where people regularly consume spices, is much lower than in countries of the western world. Extensive research over the last 10 years has indicated that nutraceuticals derived from such spices as turmeric, red pepper, black pepper, licorice, clove, ginger, garlic, coriander, and cinnamon target inflammatory pathways, thereby may prevent neurodegenerative diseases. Bioactive components of many foods added during cooking have potential antioxidant, anti-inflammatory, antimicrobial, antibacterial and chemopreventive properties. India has some of the highest spice consumption in the world, among 3,625 participants in the India Health Study. An important characteristic of cancer is that at some point in their development, they overcome the suppression of the immune system. Curcumin reverses breast cancer mediated suppression of NKC cytotoxicity. Curcumin also been shown to be very effective against malignant melanoma which normally has a response rate of only 10-15%. Curcumin induces proapoptotic effects in human melanoma cells, and modulates the cytokines. Nitric oxide (NO) production by immune cells showed an initial down regulation of Th1 cytokine response and NO production by macro-phages, and their up-regulation in NKC, which picked-up upon prolonged treatment with Curcumin, culminating in a stronger tumoricidal effect. Curcumin is a very effective chemopreventive therapeutic agent. Extensive research has addressed the chemotherapeutic potential of curcumin. Curcumin can modulate the effects interferon and interleukin, which are all important in the maturation process of NK cell activity. However, it is difficult to differentiate the actual effects of curcumin on the NK cell activity versus the direct cytotoxic effect of curcumin on tumors cells.

### Garlic

Complementary and alternative medicine (CAM) use, including herbals and multivitamin supplements, is quite common in the U.S., and has been shown to be highest in breast cancer survivors. There is an inverse association between total intake of the Allium vegetables (onions, garlic, shallots, leeks, chives, etc.) and risk of intestinal gastric cancer. The chemopreventive effects of Allium vegetables have been studied extensively in reducing the risk for cancer risk. Epidemiological observations and laboratory studies have indicated the anti-carcinogenic potential of garlic, which has been traditionally used for various human diseases around the world. Garlic (Allium sativum) holds a unique position in history and is recognized for its therapeutic potential. The health promoting properties of garlic often referred to its sulfur containing metabolites, e.g. allicin and its derivatives. Garlic is useful in preventing the suppression of immune response associated with increased risk of malignancy as it stimulates the proliferation of lymphocytes, macrophage phagocytosis, stimulates the release of in-terleukin-2, tumor necrosis factor-alpha and interferon-gamma, and enhances NKC. Aged garlic extract (AGE) has significant biological activities including immune-modulative and antioxidative effects. The number of NKC and the NK cell activity increased significantly utilizing AGE. A glycoprotein with molecular weight (MW) of about 14 kDa was isolated from garlic extract and it induced NK cell augmentation against a K562 tumor cell line.

Garlic may be useful in preventing the suppression of immune response that is associated with an increased risk of malignancy. Garlic can detoxify chemical carcinogens and may directly inhibit the growth of cancer cells. Garlic stimulates the proliferation of macrophages and lymphocytes and protects against the suppression of immunity by chemotherapy and ultraviolet radiation. NK cell cytotoxic activities were evaluated by Hassan et al. in which four garlic preparations significantly enhanced NK cell and killer cell activities of the spleen cells of tumor-bearing mice. Only AGE inhibited the growth of inoculated tumor cells. These results suggest that different types of garlic preparations have different pharmacologic properties, and among the four garlic preparations studied, AGE gave the best anticancer response. These studies strongly suggest that AGE could be a promising candidate as an immune modifier. In order to study the effect and mechanism of garlic on preventing oral precancer, 32 Wistar rats were randomly divided into two groups. The garlic group was painted with garlic solution on the hard palatal mucosae. The control group was applied with distilled water that is equal in quantity. Then, chemical carcinogen 4-nitroquinoline 1-oxide (4NQO) was painted on the same sites for both groups, three times weekly. The results revealed that garlic effectively pre-vented oral precancer induced by 4NQO. This effect may be related to the following factors that garlic can improve the activation of NKC, the function of T-lymphocytes, and the level. Fraction 4 (F4), a protein fraction isolated from aged garlic extract, enhanced cytotoxicity of human peripheral blood lymphocytes (PBL) against both natural-killer (NK cell)-sensitive K562 and NK cell-resistant M14 cell lines. Of the many beneficial actions of garlic, inhibition of the growth of cancer may be the most important. Kim, Bommareddy and Singh have shown that garlic constituent diallyl trisulfide (DATS) inhibits growth of cultured and xenografted human prostate cancer cells in association with apoptosis induction. They indicated that the DATS-induced apoptosis in prostate cancer cells is mediated in part by suppression of X-linked inhibitor of apoptosis (XIAP) protein expression, and that XIAP represents a viable biomarker of DATS. Diallyl sulfide (DAS), one of the main active constituents of garlic, causes growth inhibition of cancer cells in vitro and promotes immune responses in vivo in experimental set-tings. The study by Wu et al. revealed that DAS significantly inhibits the growth and induces apoptosis of human cervical cancer HeLa cells in vitro. S-allyl cysteine (SAC), a sulfur containing amino acid derived from garlic, has been reported to have antioxidant, anti-cancer, anti-hepatotoxic and neurotrophic activity. This study concludes that intervention of SAC could prevent free radicals associated deterioration of cognitive functions and neurobehavioral activities. Morioka et al., found that F4 is a very efficient immune-potentiator and may be used for immunotherapy. Allicin, the main flavor compound in garlic, has anti-carcinogenic activities in a range of cancer cells. DATS is a garlic organosulfide that is toxic to cancer cells. At the doses evaluated, DATS appears to be an effective attenuator of BaP-induced breast carcinogenesis, in vitro. Diallyl disulfide (DADS) is a major organosulfur compound derived from garlic, gastric adenocarcinoma cell line at various concentrations. DADS decrease the viability of AGS cell lines and induce apoptosis in a dose-dependent manner. SAC was identified as a potent compound de-rived from garlic. This study demonstrated the chemo preventive activity of SAC in vitro, and that SAC may be a promising candidate for prostate cancer treatment in which natural allyl sulfur compounds show antiproliferative effects on tumor cells.

Garlic extracts exert anti-cancer and anti-inflammatory effects through antioxidant mechanism. Morihara et al. examined the effectiveness of AGE, a garlic preparation rich in water-soluble cysteinyl moieties, and its component for scavenging of superoxide by using the hypoxanthine-xanthenes oxidase and human neutrophils. Aged garlic extract (3 mg/ml) significantly inhibited superoxide production, suggest that aged garlic extract may be useful for preventing diseases associated with reactive oxygen species. Their studies showed that DATS induced apoptosis in human colon cancer HT29 and in colon 205 cell lines in vitro. Findings indicated that DATS inhibits tumor growth in an allograft animal model. Thus, DATS may represent a colon cancer preventive agent and can be used in the future. Medicinal benefits of Allium vegetables, such as garlic, have been noted throughout recorded history, including protection against cancer and cardiovascular disease. Powolny et al. data suggest that DATS is able to increase worm lifespan by enhancing the function of the pro-longevity transcription factor skn-1. The antitumor properties of water-soluble sulfur compounds may play a relevant role in the formation of both reduced glutathione conjugates and radical species that affect the activity of the thiol-proteins involved in fundamental cellular processes. Report showed that the aqueous garlic extract S-allylcysteine (SAC) inhibited the epithelial-mesenchymal transition (EMT) of human oral cancer CAL-27 cells in vitro. It was shown that SAC has potential as an agent against tumor growth and the progression of oral cancer.

Hydrogen sulfide (H₂S) is a novel gasotransmitter that regulates cell proliferation and other cellular functions. Sulforaphane (SFN) is a sulfur-containing compound that exhibits anticancer properties, and young sprouts of broccoli are particularly rich in SFN. A H₂S-releasing diet or drug might be beneficial in the treatment of prostate cancer.

There is mounting evidence that aged black garlic extracts (ABGE) possess significant anticancer actions. The anti-cancer action of ABGE may be partly due to its antioxidant and immune-modulative effects. A major contributing factor to the prevention of colon cancer is in the use of dietary constituents, such as Garlic oil to prevent the development of colorectal cancer. Garlic phytochemicals influences the pharmacokinetic and pharmacodynamic behavior of concomitantly ingested drugs. Garlic consumption not only causes increased energy demand from the faster RBC turnover but also increases the production of CO, a pleiotropic gasotransmitter, which in turn stimulates splenic erythropoiesis by an erythropoietin-independent mechanism, thus completing the sequence of feedback regulation for RBC metabolism. Being a pleiotropic gasotransmitter, CO may be a second messenger for garlic's other physiological effects. The scavenging capacity of reactive oxygen species, such as hydroxyl radicals, is reported not to decrease in boiled garlic. These findings indicate a promising potential for the use of garlic-derived sulfur compounds in chemoprevention and chemotherapy. Garlic and garlic-derived compounds reduce the development of mammary cancer in animals and suppress the growth of human breast cancer cells in culture. Ma et al. reported that 14.7-year follow-up for gastric cancer incidence and cause-specific mortality among 3365 randomly assigned subjects in this masked factorial place-bo-controlled trial. Garlic and vitamin treatments were associated with non-statistically significant reductions in gastric cancer incidence and mortality. Vitamin treatment was associated with statistically significantly fewer deaths from gastric or esophageal cancer, a secondary endpoint (p=0.014). Health benefits of garlic and other Allium vegetables (e.g., onions), such as lipid lowering and anticancer effects, are credited to metabolic byproducts, including DATS. Known pharmacological responses to this natural product include alteration in carcinogen-metabolizing enzymes, cell cycle arrest, induction of apoptotic cell death, suppression of oncogenic signal transduction pathways, and inhibition of neoangiogenesis. The mechanism of biological action of garlic-derived sulfur compounds in human hepatoma (HepG2) cells can be dependent on the presence of labile sulfane sulfur in their molecules, thereby, influencing transmission of signals regulating cell proliferation and apoptosis.

Fresh garlic has the highest content of bioactive components and the greatest anticancer efficacy. Walter et al. indicated that the use of garlic and grape seed may be associated with reduced risk of hematologic malignancies. S-allylmercaptocysteine (SAMC), one of the water-soluble organosulfur garlic derivatives, has been demonstrated as a suppressive agent against some tumors. The effects of SAMC on the proliferation and metastasis of colorectal cancer (CRC) under in vitro and in vivo conditions were evaluated.

Taken together, the proliferation and metastasis of CRC cells can be significantly suppressed by SAMC treatment under both in vitro and in vivo conditions. SAMC may thus be a promising candidate for CRC chemotherapy. The Allylmercapto glutathione S-conjugate, S-allylmercapto-L-cysteine (SAMC) is biotransformed from allyl sulfides and from naturally occurring water-soluble garlic derivatives. The results from Lee et al. suggested that SAMC may regulate bcl-2 and baxto induce apoptosis in transplanted tumor cells. Further studies should be conducted to establish the clinical expediency of malignant neuroblastomas in humans.

Diallyl disulfide (DADS), a sulfur compound derived from garlic, has been shown to have protective effects against colon carcinogenesis in several studies performed in rodent models. DADS showed anti-proliferative effects on colon cancer HT-29 cells, and DHDG and DHUG genes were found to be involved in this process. SAMC-induced apoptosis is associated with the Id-1 pathway, and that the inactivation of Id-1 enhances the ability of SAMC to inhibit the survival, invasion and migration of bladder cancer cells.

Garlic reduces the side effects caused by anti-cancer agents. Thus, garlic and garlic-derived compounds are promising candidates for breast cancer control. The ability of garlic preparations to inhibit cancer cell-growth has been attributed to a group of structurally-related organosulfur compounds found in the crushed clove. For many years, in vitro and in vivo studies have reported that organosulfur compounds (OSCs), naturally found in Allium vegetables are able to suppress the proliferation of various tumor cells. Furthermore, growth of both sensitive (MCF-7) and resistant (Vcr-R) human breast carcinoma cells was significantly decreased by these tetrasulfides. The observed antiproliferative effect appeared to be associated with a G2-M cell cycle arrest. There is a body of evidence showing that several lifestyle and dietary factors are associated with colorectal polyps' formation. Garlic has been valued in many cultures both for its health effects and as a culinary flavor enhancer. Garlic's chemical complexity is widely thought to be the source of its many health benefits, which include, but are not limited to, anti-platelet, pro-circulatory, anti-inflammatory, anti-apoptotic, neuro-protective, and anti-cancer effects. While a growing body of scientific evidence strongly upholds the herb's broad and potent capacity to influence health, the common mechanisms underlying these diverse effects remain disjointed and relatively poorly understood. Boiled garlic powder (BGP) may exert chemopreventive effects against colon carcinogenesis at least in the initiation stage. Epidemiological studies iNKC increased garlic consumption with a reduced incidence of cancer in various human populations.

Garlic also induced apoptosis, and this was associated with an increase in caspase3 activity. The association between diet and prostate cancer (PC) risk, although suggestive, still remains largely elusive particularly in the Asian population. In 194 cases with PC and 317 controls, that total fat may increase PC risk and tomatoes/tomato products and garlic may protect patients against PC. Cardiovascular diseases and cancer are the leading causes of death in most countries. These diseases share many common risk factors as well as pathogenetic determinants, and their incidence is related to age in an exponential manner. Mechanisms responsible for the cardiotoxicity of anticancer drugs, the possibility to prevent them and provide examples of diet-derived phytochemicals and other biological substances that could be exploited for protecting the cardiovascular system according to a joint cardio-oncological preventative approach.

Alzheimer's disease (AD) is the most common form of dementia in the older people and 7th leading cause of death in the United States. Thus, based on the reported positive preliminary results reviewed, further research is required to develop the full potential of ‘Aged Garlic Extract’ (AGE) and/or SAC into an effective preventative strategy for AD. Diallyl sulfide (DAS), diallyl disulfide (DADS), and diallyl trisulfide (DATS) are major organosulfur compounds exiting in garlic. These studies demonstrate that application of DAS, DADS, and DATS might serve as potential antimetastatic drugs. There is strong evidence from studies of prostate cancer cell lines and rodent models that several garlic derivatives may have anti-inflammatory, antioxidant, or other anticancer proper-ties. These derivatives include DAS, DADS, DATS and ajoene which show these to be promising anticancer agents in oncohematology.

OSCs derived from plants, fungi or bacteria can serve as chemopreventive and/or chemotherapeutic agents and have been attracting medical and research interest as a promising source for novel anti-cancer agents. Amphotericin B (AmB), a classical antifungal drug is limited by its side effects and the emergence of drug-resistant strains. The enhancing effect of allicin is dependent on the inhibition of ergosterol-trafficking from the plasma membrane to the vacuole membrane, which is considered to be a cellular response to protect against disintegration of the vacuole membrane. Allicin found in crushed garlic acts synergistically with AmB and the synergistic fungicidal activities of AmB and allicin may have significant implications for the development of vacuole-targeting chemotherapy against fungal infections.

### Genistein

Genistein (GEN), the active component of Soy, stimulates antiangiogenesis components, and its estrogen like structure would warrant it having immune modulating activity that would involve NK cell stimulation. Ex-posure to GEN in a rat model increased the percentages helper and cytotoxic T cells. Epidemiological studies have suggested that populations with high isoflavone intake through soy consumption have lower rates of breast, prostate, and colon cancer. Isoflavone GEN in soybean is considered a potent chemopreventive agent against cancer as copper levels are known to be considerably elevated in almost all types of cancers. Ullah et al. in 2011 showed that GEN is able to target endogenous copper leading to pro-oxidant signaling and consequent cell death and explains why GEN has a preferential cytotoxicity towards cancer cells.

Furthermore, GEN is considered to exert potent anti-tumor effect partially through its anti-angiogenesis property. GEN exerts its anti-cancer effects by inhibiting protein tyrosine kinase that is involved in up-regulation of VEGF. Zhou et al. in 2012 provided an experimental basis for combined use of ATRA and GEN in the treatment of lung cancer. The inhibition of protein tyrosine kinase (PTK) activity and MAPK activation and the de-crease in matrix metalloproteinases (MMPs) production and activity by GEN interrupt VEGF-stimulated endothelial cell activation, which thereby may represent a mechanism that would explain the anti-angiogenesis effect of GEN and its cancer-protective function. Genistein aglycone might be an effective alternative therapy for the management of age-related skin changes in postmenopausal women. GEN reduced human colon cancer cell line DLD-1 cell viability and increased apoptosis.

In breast cancer, the interaction between estrogen-producing breast adipose fibroblasts (BAFs) and estrogen-dependent epithelial tumor cells is pivotal. Local estrogen production is catalyzed by aromatase, which is differentially regulated in disease-free and tumorigenic breast tissue. The use of aromatase inhibitors to block local estrogen production has been proven to be effective in treatment of estrogen-dependent breast cancer. Worldwide geographical variation in cancer incidence indicates a correlation between dietary habits and cancer risk.

In several cancer types, expression of multidrug resistance (MDR) proteins has been associated with lack of chemotherapy response. In advanced prostate cancer (PCa) the use of chemotherapy is mainly palliative due to its high resistance. Soy consumption is associated with a lower incidence of colon cancer. These novel anti-proliferative mechanisms of GEN suggest a possible role of combining GEN with other chemoreceptive agents for the treatment of colon cancer. GEN possesses a structure similar to estrogen and can both mimic and antagonize estrogen effects although at high concentrations it inhibits breast cancer cell proliferation.

GEN possesses anti-cancer, anti-inflammatory, anti-oxidant and anti-osteoporosis effects that may be a promising drug to inhibit the inflammatory process and prevent liver damage in patients with non-alcoholic steatohepatitis. Cancer cells require high levels of lipid synthesis to produce structural, signaling and energetic lipids to support continuous replication. Lipogenesis can occur mainly by the tandem activation of acetyl-CoA carboxylase, fatty acid synthase and stearoyl-CoA desaturase-1. Metabolic pathway is a potential anti-cancer target for nutritional and pharmacological interventions by globally down regulating lipid biosynthesis. GEN suppresses cancer cell growth, emphasizing the relevance of this botanical compound as a potential therapeutic agent against lung cancer, a disease for which therapeutic choices remain limited. Up regulation of Brca1 may be required for prepubertal dietary genistein exposure to reduce later mammary tumorigenesis, per-haps because in the absence of this up regulation, mice do not exhibit GEN-induced downregulation of estrogen receptor (ER-α), progesterone receptor (PgR) and RaNKCl. Various chemopreventive compounds alter gene expression, possibly explaining their biological activity. The effective activity of GEN on osteolytic bone metastasis and bone mineral was investigated. GEN could inhibit osteolytic bone metastases, suppress bone resorption, increase bone mass and improve bone micro-structure in bone metastases of breast cancer. Genistein and methoxychlor modulate the activity of NKC and the expression of phenotypic markers by thymocytes and splenocytes in a rat model. Adult T-cell leukemia (ATL) occurs in human T-lymphotropic virus type I-infected individuals and is endemic to the southwestern area of Kyushu in Japan. GEN had cytotoxic effects on ATL cells. Whether these anticancer activities involve modulation of the NK cell remains to be investigated.

### Ginseng

An Acidic Polysaccharide of Ginseng (APG), so called ginsan, is a purified polysaccharide. APG has multiple immunomodulatory effects of stimulating NKC and T cells and producing a variety of cytokines that diminish the proinflammatory response. APG may serve as an effective therapy for human multiple sclerosis (MS) and other autoimmune diseases. Ginseng is widely used in Asian countries as a tonic to increase energy; however its efficacy for treating cancer-related fatigue has not been carefully studied. Ginseng polysaccharide has certain improving immune function effect in nasopharyngeal carcinoma and in patients during radio-therapy (RT); it could also eliminate the occurred adverse reaction to RT and the general condition of patients.

Ginsenoside Rh2, isolated from an ethanol extract of the processed root of Panax ginseng CA Meyer, inhibits the growth of B16 melanoma cells in induction of apoptotic cells in the tumor and increases NK cell activity in spleen cells from tumor-hearing nude mice. Panax ginseng was shown to stimulate cellular immune function by peripheral blood mononuclear cells (PBMC) from normal individuals and patients with either the chronic fatigue or the acquired immunodeficiency syndrome. Furthermore, ginseng enhances cellular immune function of PBMC both from normal individuals and patients with depressed cellular immunity. In 63 patients with stomach cancer, the results showed that the count of T lymphocytes in chemotherapy combined with Shenmai (ginseng preparation) injection (SMI) group increased, while in control groups it decreased. Also, activities of NK cell and lymphokine activated killer cell (LAK) level increased in the SMI group. The ethanol-insoluble fraction of ginseng was tested for immunomodulatory activity. These results suggest that the ethanol-insoluble fraction of ginseng shows antitumor effects by being immunomodulator of NK cell activity. The positive effect of Panax ginseng extracts on cell-mediated immune functions in men has been shown in randomized trials. The immunomodulatory activity of ginsenoside increased the number of T-helper cells with respect to the whole T-cell number and increased the splenocyte NK cell activity. The pharmacological effect of ginseng is its ability to replenish vital energy, strengthen the body's immune resistance and improve memory.

### Lentinan

As reviewed by Wasser, the species of mushrooms on Earth is estimated at 140,000, yet maybe only 10% are known. Mushrooms comprise a vast and yet largely untapped source of powerful new pharmaceutical products. In particular, and most importantly for modern medicine, they represent an unlimited source of polysaccharides with antitumor and immunostimulating properties. Data on mushroom polysaccharides have been collected from 651 species and 7 infraspecific taxa from 182 genera of higher Hetero- and Homobasidiomycetes. These polysaccharides are of different chemical composition, with most belonging to the group of beta-glucans; these have beta-(1→3) linkages in the main chain of the glucan and additional beta-(1→6) branch points that are needed for their antitumor action. High molecular weight glucans appear to be more effective than those of low molecular weight. Most of the clinical evidence for anti-tumor activity comes from the commercial polysaccharides Lentinan (LEN). LEN is especially beneficial in clinics when used in conjunction with chemotherapy. Mushroom polysaccharides prevent oncogenesis, show direct antitumor activity against various allogeneic and syngeneic tumors, and prevent tumor metastasis. Polysaccharides from mushrooms do not attack cancer cells directly, but produce their antitumor effects by activating different immune responses in the host. The antitumor action of polysaccharides requires an intact T-cell component; their activity is mediated through a thymus-dependent immune mechanism.

LEN, a polysaccharide extracted from a kind of black mushroom shiitake, has been clinically applied as an antitumor and antimetastatic drug, and has been reported to prevent both chemical and viral carcinogenesis. It is known that LEN affects the tumorous vascular system resulting in the induction of hemorrhagic necrosis which is dependent on T-cells in the tumor.

LEN, an antitumor polysaccharide used clinically in Japan, requires the intact T cell compartment to manifest its antitumor effects. These results suggest that, in addition to the augmentation of immune effectors cell activity against tumors, infiltration of these cells into the tumor, initiated by the delayed-type hypersensitivity DTH responses at tumor sites, may be involved in eradication of tumors by LEN. NK cell-mediated killing of tumor cells both in vitro and in vivo. In a combined study, Vetvicka et al. in 2008 suggested that PS3 a sulfated derivative of a (1→3)-beta-D-glucan is similarly effective as native non-sulfated (1→3)-beta-D-glucan and is generally more active than LEN. LEN could enhance the anti-tumor immuno-reaction of Dendritic Cell Vaccine (DCV) effectively.

Glucans have a long history as nonspecific biological modulators. A novel glucan-Phycarine was isolated from sporophytes of Laminaria digitata. Phycarine showed significant stimulation of phagocytic activity as well as potentiation of synthesis and release of IL-1, IL-6 and TNF-alpha. In addition, Phycarine increased NK cell-mediated killing of tumor cells both in vitro and in vivo while acting via complement type 3 receptors. Significant regression in tumor formation was observed in LEN fed mice compared to control (unfed) mice when K36 or human colon-carcinoma cells were used. Anti-tumor property of LEN was maintained with oral administration. In addition, “primed” lymphocytes, when given passively to immunodeficient mice, were able to retard the development of tumors in these mice. The pre-operative administration of LEN for patients undergoing cardiopulmonary bypass (CPB) ameliorated the impairment of NK cell activity and promoted the rapid recovery of CD4-positive cells.

The immunopharmacological characteristics of angelan, a polysaccharide purified from Angelica gigas Nakai, was investigated in relation to the specificity to immune cells. The treatment of angelan increased the expression of IL-2, IL-4, IL-6, and IFN-gamma. The experiment of host resistance to syngeneic tumors also showed that angelan potentiated the immune functions. Han et al studied the action of Polyporus polysaccharide (PPS), mycobacterium polysaccharide (MPS) and LEN on lymphokine-activated killer (LAK) cell activity in vitro. The three combined enhanced the LAK activity by 42%-56.9%, and reduce the dose of rIL-2 needed to have the same LAK activity by 50% (P<0.01). Therefore, PPS, MPS and LEN could be used as bioactivity regulators in LAK cell therapy in tumor treatment. Rafique and Adachi concluded that the intraportal administration of chemotherapeutic agents reduces NK cell activity in the liver and the addition of LEN prevents this reduction.

The usefulness of lymphocyte subset change as an indicator for predicting survival time and the effectiveness of combined treatment with an immunopotentiator, LEN, and carboplatin (CBDCA) were investigated. Changes of lymphocyte subsets of killer T cell/suppressor T cell and NK cell on posttreatment compared to pre-treatment are clinically useful indicators for predicting and monitoring the effectiveness of combined treatment with LEN. Twenty-one patients with malignant peritoneal or pleural effusions of gastric carcinomas were treated with intracavitary injection of LEN in which LEN enhanced the induction of cytotoxic T-lymphocytes. KRN7000, a compound with a novel alpha-galactosylceramide structure significantly prolonged the lifespan of mice demonstrating that KRN7000 would be a useful agent for cancer therapy. The effects of orally administered biological response modifiers (BRMs) in preventing postoperative micro liver metastasis of primary colorectal cancer were examined in experimental animals. LEN suppressed liver metastasis and prolonged the survival period. It elevated the liver NK cell and liver macrophage activities. The life-prolonging effect of the combination of LEN and IL-2 is mediated by antigen-specific T cells and that the combination of pre- and post-operative therapy with LEN and IL-2 may be effective to prevent cancer recurrence and metastasis after surgical resection. Suzuki et al. in 1994 has indicated that the antitumor effects of LEN/IL-2 are mediated by CD8+ CTL but not by CD4+ T cells or NKC1.1+ NKC/LAK cells, and suggest that this combined therapy may be effective against even established tumors that are resistant to IL-2 therapy. LEN markedly pre-vented cyclophosphamide (CY)-induced suppression and promoted the recovery of IL-2-production. LEN exhibits lightening or restorative effects on CY-induced suppression of antitumor effectors activities of murine spleen cells. Thus it was shown that LAK cells stimulated with IL-2 plus Lentinan had strong cytotoxicity and might be useful as effect or cells for adoptive immunotherapy Tani et al.

In a study of 15 patients with gastric carcinoma, peripheral blood mononuclear cells (PBM) were obtained serially before and 3, 5 and 7 days after LEN administration. NK cell activity was significantly enhanced 7 days after the drug injection. The anti-tumor effect of LEN is due to the activation of killer cells in vivo, because the optimal concentration of LEN for the induction of killer cells in vitro was equivalent to the plasma concentration obtained after clinical doses of this agent. Lymphocyte subpopulations of the spleen were assayed in 26 patients with gastric cancer and 5 patients with benign disease using two-color flow cytometric analysis. The above results suggest that lymphocyte subpopulations in the spleen may have more immunosuppressive potential in proportion with the stage of gastric cancer, but that this reduced immune state may be altered when LEN was given to these patients. When gastric cancer patients were pretreated with BRM, such as Lentinan, LAK cells from PBMC showed higher NK cell and LAK activities as compared with those of patients without BRM pretreatment. Twenty effusions in sixteen patients with malignant peritoneal and/or pleural effusions were treated with intracavitary injection of LEN in which lymphokine activated killer activity were also augmented or maintained after LEN injection. The usefulness of Lentinan, as an agent for post-operative adjuvant therapy, was investigated in patients with gastrointestinal cancer. LEN had a marked immunopotentiating efficacy in the stage IV gastrointestinal cancer patients. There is clinical application of a combination of LEN and IL-2 for immunotherapy against cancer without detrimental side effects. The adoptive transfer of LAK cells combined with intraperitoneal administration of both recombinant interleukin-2 (rIL-2) and LEN markedly inhibited the growth of the endometrial tumor cell line HHUA xenografts in nude mice, while no response was observed in nude mice treated with LAK cells plus either recombinant (rIL-2) or LEN, or treated with rIL-2 plus LEN alone.

These results suggest the clinical application of adoptive immunotherapy in association with LAK cells, rIL-2, and LEN as a treatment of gynecologic cancers. These results also indicate that intrapleural Lentinan administration is a useful treatment for carcinomatous pleuritis and peritonitis. IL-2 and LEN in combination are more effective than either one alone for inducing destruction of pulmonary metastases. Endoscopic intratumoral injection of LEN may enhance local and systemic immunity in patients with gastric cancer. The in vitro effects of LEN on NKC, antibody-dependent cell-mediated cytotoxicity (ADCC), lectin-dependent cell-mediated cytotoxicity (LDCC) and mitogen-induced blast transformation were studied in patients with solid tumors and chronic lymphocyte leukemia (CLL). LEN increased natural cell-mediated cytotoxicity (NCMC) of tumor-bearing subjects. The most prominent enhancement of NK cell and ADCC activity was seen in CLL patients, where a dose-related increase was seen (from 0.01 to 1 microgram/ml). Gastric cancer patients treated with chemotherapy with or without LEN, were showed excellent end-point results only in Lentinan-administered patients with normal protein levels was seen.

The interferon level in the peripheral blood circulation of cancer patients was elevated 12 h following LEN administration, and NK cell activity of peripheral mononuclear cells was enhanced in 48 h. These data indicate that LEN favorably affects the host mechanisms of man. In patients with cancer of digestive organs, NK cell activity and PHA induced blast response for lymphocytes increased significantly. Cancer patients (n=11) received LEN i.v. 2 mg, NK cell activity of lymphocytes was increased in four patients on the following day. These results concluded that LEN is promising as a new type of polysaccharide immune-adjuvants useful for cancer chemotherapy where chemotherapy alone has shown little anti-tumor activity against advanced oral squamous cell carcinoma (OSCC) patients.

LEN administration was shown to be effective against OSCC and has the potential of being a new therapeutic tool for future treatment of these tumors. Combination of LEN with standards of care in acute myeloid leukemia, idarubicin, and cytarabine increased average survival compared with monotherapy and reduced cachexia. LEN is efficient in promoting chemosensitivity in colon carcinoma cells, has much less side effects, and has great potential to be applied as a supplement for colon cancer therapy.

Complementary alternative medicine is actively per-formed as a cancer therapy supplementary for food containing superfine dispersed LEN (beta-1,3-glucan) in patients with unresectable or recurrent hepatocellular carcinoma in a multi-center study. In a 36 patient survival times of Lentinan-high-binding group were significantly longer than those of Lentinan-low-binding group. LEN is effective for hepatocellular carcinoma patients' survival especially when long-time ingestion is implemented. Assessment of Lentinan-binding CD14+ monocytes is a promising prognostic predictor. Glucans have a long history as nonspecific biological modulators and are involved in the effects of three different glucans on immune reactions such as the synthesis and release of interleukin (IL)-1, IL-2, IL-4, IL-6, IL-8, IL-13, and tumor necrosis factor-alpha. In addition, a LEN derivative inhibited growth of tumor cells in vivo and affected expression of several important genes in breast cancer cells. LEN CD8-positive cells appeared to have suppressed tumor cell proliferation. CD4-positive cells appeared to be involved in this activity of CD8-positive cells. LEN has a life prolonging effect in non-operable, recurrent gastric cancer patients in combination with chemotherapy.

Dendritic cells DCs might play an important role in chemotherapy, and LEN presents a promising chemo immunotherapy, which might lead to better survival for cancer patients. Yoshino, et al. demonstrated that patients with advanced cancer may have impaired cell-mediated immunity caused by an imbalance between Th1 and Th2 responses. LEN can cancel Th2-dominant condition in patients with digestive cancers and may improve the balance between Th1 and Th2. LEN may augment the effects of erythropoietin (Epo) on erythropoiesis in the course of anemia and the decreased erythropoiesis in cancer patients receiving chemotherapy. Multiple biological therapies are considered to be potentially effective against the highly malignant advanced tumors.

### Mistletoe

NKC play an important role in cell-mediated immune responses against tumor cells. Strengthening the immune system might help to control or even to eradicate malignant tumors. Insights into the mechanisms of host-defense against cancer cells and the modification of immune-effector cells might pave the way to efficient treatment strategies, even in the preventive application setting.

As reviewed by Braedel-Ruff in 2010, extracts produced from Viscum album L. (mistletoe), they are widely used in complementary medicine for the treatment of cancer. In many preclinical and clinical studies, viscum album extracts were shown to exert immunomodulatory functions. The NKC are involved in direct tumor cell destruction. There is evidence that they may contribute to the prevention of the control of metastases. Twenty three tumor patients were treated subcutaneously with in-creasing concentrations of aqueous mistletoe extracts. The relative amount of lymphocytes and the number of NK cell increased significantly. Non-selected tumor patients (n=12) with various solid carcinomas were treated continuously twice weekly over 48 weeks with the aqueous mistletoe extract with a statistically significant rise in the cell count of total lymphocytes, monocytes and NKC. The raised cell count of NKC correlated with the significantly increased cytotoxic activity versus tumor cells. Other lymphatic subpopulations, for instance CD3+, CD8+, CD3+CD4+ and CD3+CD8+ cells, also revealed distinct rise in cell count in the course of treatment. Within 6 weeks after completion of treatment, the overall values dropped again. NKC were still raised in comparison to the baseline values. These studies showed a significant improvement in the immune status of tumor patients. It has shown to be safe over a long period. Breast cancer patients (n=20) received a single intravenous infusion of Iscador, a mistletoe (Viscum album L.) extract. NK cell and antibody-dependent cell-mediated cytotoxicity (ADCC) activities as well as the number of large granular lymphocytes (LGL) showed significant increases in NKC/ADCC activities and LGL frequencies were observed. Cultures of human PBMC as well as cultures of preseparated peripheral non-adherent cells (PNAC) and monocytes showed enhancement of NK cell cytotoxicity against K562 tumor cells. The mistletoe lectins have been identified as the main active principle of mistletoe extracts. They have been shown to exhibit induction of cytokine secretion and increased activity of NK cell. Clinical application of mistletoe extracts or isolated lectins is reported to induce number and activity of NKC in peripheral blood in a dose-dependent manner. Mistletoe lectins (MLs) is specific for galactose, high-density galactose expression in malignant melanoma is a predictor of poor prognosis.

### N-Acetyl-Cysteine

N-Acetyl-Cysteine (NAC) is a natural, in vivo derived antioxidant that induces modulation of glutathione (GSH) cellular content effects. The levels of GSH are decreased significantly in NKC derived from individuals with HIV infection. Downregulation of class I by lactacystin (LAC) significantly enhances NK cell-mediated lysis of multiple myeloma (MM). Furthermore, the downregulation degree of class I was associated with increased susceptibility of myeloma cells to NKC killing. An important role for NK cell in the regulation of T-cell responses is emerging, although the receptor pairs regulating the NKC-T-cell interaction have still not been identified. Impaired poliovirus receptor (PVR) is reduced in the presence of the reactive oxygen species (ROS) scavenger NAC. NKC lyses allogeneic responses were efficient with NAC. Gamale\check{i} studied NAC ability to change the phenotype properties of several transformed and embryonic cells. It demonstrated that treatment with NAC (10 mM) results in a loss of susceptibility to NK cell activity. Murine embryonic fibroblasts (MEFs) pretreated with 10 mM NAC as well as transformed cells lose their susceptibility to NK cell activity. The loss of cell sensitivity to NK cell cytolytic activity was accompanied by a reorganization of the actin cytoskeleton and the appearance of well-pronounced stress.

Alpha-lipoic acid (ALA) and NAC showed an increase sensitivity of 3T3-SV40 fibroblasts to lytic activity of NKC. ALA reduced significantly the fibroblast sensitivity in several hours, whereas NAC (10 mM) did not change it. Both NAC and ALA pathway includes inactivation of matrix metalloproteinase (MMP-2). NAC is a known antioxidant and induces modulation of glutathione cellular content effects. Usage in stem cell transplantation (SCT) may be relatively safe with regard to the graft-vs.-leukemia effect (GVL) effect, yet further clinical studies are warranted. 18-20 h cultivation of transformed mouse fibroblasts 3T3-SV40 in the presence of antioxidant, NAC (NAC, 10 mM), did not change their sensitivity to lysis by NKC mouse splenocytes. However, in 18-20 h after NAC removal 3T3-SV40 cells demonstrated resistance to NK cell activity. This effect may be due to the NAC-induced modifications of the cell surface and extracellular matrix proteins. Antioxidants are able to protect umbilical blood mononuclear cells against oxygen stress and affect oxygen stress mediated immune function inhibition of umbilical blood.

With aging there is an increase of oxidative stress due to an imbalance between the oxidant production and the antioxidant levels in favor of the former. Since immune cell functions are specially linked to ROS generation, the oxidant/antioxidant balance is essential for these cells. The ingestion of a diet supplemented with two thiolic anti-oxidants, such as NA cell and thioproline (TP) has been shown to be beneficial to the immune response in prematurely aging mice (PAM). The ingestion of very high amounts (0.3%) of thiols by aged animals increased the phagocytosis, the NK cell activity and especially the lymphoproliferative response to the mitogen, a function that is very depressed with aging. With aging there is an increase of oxidative stress due to an imbalance between the oxidant production and the antioxidant levels in favor of the former. Since immune cell functions are specially linked to ROS generation, the oxidant/antioxidant balance is essential for these cells. The ingestion of thiols by aged animals increased the phagocytosis, the NK cell activity and specially the lymphoproliferative response to the mitogen, a function that is very depressed with aging. These results add new insights to the key role played by redox imbalance as a modulator of immune system homeostasis and suggest that an antioxidant drug such as NAC could be useful against pathologies associated with an increase in lipid peroxidation.

The induction of endogenous glutathione in living cells immediately after low-dose gamma irradiation is at least partially responsible for the appearance of enhanced NK cell activity. Interleukin-12 (IL-12) is secreted from monocytes and macrophages; it exerts pleiotropic effects on T cells and NK cell, and stimulates IFN-gamma secretion. IFN-gamma and IL-4 oppositely affect the glutathione (GSH)/(GSSG) glutathione balance, which may regulate IL-12 secretion from AM in response to LPS.

Massive loss of sulfur in HIV-infected subjects and the present demonstration of the immunoreconstituting effect of cysteine supplementation indicate that the HIV-induced cysteine depletion is a novel mechanism by which a virus destroys the immune defense of the host and escapes immune elimination. Two randomized placebo-controlled trials have shown that treatment of HIV-infected patients with NAC caused in both cases a significant increase in all immunological functions under test, including an almost complete restoration of NK cell activity. This stimulation of the NK cell activity by antioxidants is an important favorable response, especially in old mice, in which age results in a decrease in NK cell function and, therefore, in a higher incidence of neoplasia. Suppression of calcineurin and NFAT activation is a mechanism by which oxidative stress inhibits FasL induction in activated NKC. Thiol-reducing compounds, such as NAC, are required for maintenance of optimal NKcell functions under physiologic oxidative conditions. NAC raises GSH levels and increases cytotoxicity. Hepatic GSH is important to the optimal functioning of the hepatic immunity that protects against hepatoma development.

cell cycle progression is controlled in human NK cell through redox regulation of expression and phosphorylation of retinoblastoma gene product protein using NAC. Fas-sensitive cells were incubated with NAC, intracellular GSH was increased and Fas-mediated apoptosis was blocked. Mediated apoptosis, but not perforin-dependent killing, is modulated by intracellular intracellular glutathione (GSH) in human T lympho-cytes. NAC is capable of enhancing the adhesion properties of the epithelial cell line. NAC could thus behave as a drug influencing certain cytoskeleton-dependent cell processes in a non-histotype dependent manner. Importance of intracellular oxidative balance on cell-mediated cytotoxicity was performed by analyzing the effects of the NAC, on NKC-mediated cytotoxicity. The results obtained indicate that an enhancement of target cell (TC) killing can be detected when a pre-exposure of effector cells (EC) to NAC was performed. NAC may be useful as an adjunct to increase the antitumor activity of IL-2/LAK therapy. Anti-tumor effect of haemin and IL-2 was enhanced (63% decrease in metastases) by administration of NAC. Since haemin can safely be administered to patients, it may represent a new class of biologic response modifiers that could enhance IL-2-mediated anti-tumor effects.

NAC has ability to change the phenotype properties of several transformed and embryonic cells. The loss of cell sensitivity to NK cell cytolytic activity was accompanied by a reorganization of the actin cytoskeleton. The results obtained demonstrated that the direct effect of NAC (having reduced thiol groups), and the indirect one, ALA (reducing thiol groups and acting as a direct antioxidant only inside the cell) activate principally different intra-cellular signal pathways. However, both NAC and ALA pathway includes inactivation of MMP-2 resulting in a strong synergistic antiangiogenic activity. NAC mildly inhibits the graft-vs.-leukemia effect but not the LAK activity. Intracellular oxidative balance on cell-mediated cytotoxicity was performed by analyzing the effects of NAC. NAC as a cytoskeleton thiol-modifier contributed to the activation of effector cells.

### Resveratrol

For almost 200 years, scientists have suspected that wine consumption had something to do with “The French Paradox”—the fact that 50% more Frenchmen live to 100 than America and other countries, despite a rich, high fat diet. But only in recent years has the explanation become clearer. The polyphenols, such as Resveratrol (RES) found in red wine may slow down the aging process and help prevent age-related diseases including heart disease, cancer, Alzheimer's, diabetes, and others. RES and vitamin C combination is a strong activator of phagocytosis and antibody formation. Two different models of cancer treatment demonstrated that the combination strongly suppressed the growth of breast and lung tumors, most likely due to the stimulation of apoptosis.

RES can block cell proliferation and induce growth arrest and/or cell death in several types of cancer cells. The effect of RES on the induction of apoptosis via autophagy is mediated through ROS in human colon cancer cells, such as the combination of dietary supplements such as RES, Lycopene, Vitamin C and Anthocyanins. RES also reduced skin toxicity due to external beam radiotherapy in patients affected by breast cancer. Interactions between various dietary cancer chemopreventive phytochemicals in drug transporter functions are not well studied, for example the effects of genistein and RES on the multidrug resistance protein 2 (MRP2) expressions. It can potentially interfere with each other's regulatory function on the cancer chemoprevention-related genes through a competitive mechanism. RES down-regulates protein S expression in HepG2 cells. RES has a variety of activities involved in the immune system and in longevity. There are number of anticancer mechanism for resveratrol including modulation of the immune system through chemo preventative means. It can even function as a prooxidant against cancer cells as a dietary chemo preventative agent, thus earning the title as a so called longevity drug. The cytotoxic effect has been shown to destroy a number of organ based cancers including colon cancer, pancreatic cancer, ovarian cancer, and gastric cancer and glioma cells. It even diminishes the effects of cis platinum cytotoxicity. In vivo cytotoxic activity of the NKC isolated from resveratrol-pretreated rats is enhanced compared with that of the non-pretreated rats. Resveratrol may act, like the IL-2 pathway, to enhance perforin expression and cytotoxic activity. RES modulates several human immune cell functions and suggests that this activity may be related to its effects on cytokine production by both CD4+ and CD8+ T cells. The mechanisms RES is used to destroy cancer cells include increment in NK cell activity NKC by direct cytotoxicity and induced apoptosis in target cells using the perforin and granulozyme activity.

### Selenium

Selenium (Se) has been recognized for years as an essential trace element for animals (National Research Council, 1980). It is generally accepted that Se is necessary for optimum performance of the immune system. Selenium deficiency results in immune suppression but little is known concerning the effect of excess Se on immune function. Recent evidence suggests that oral Se supplementation may impede oncogenesis. Antioxidant supplementation with Se may be useful for the preservation of health and functional longevity in aging populations. Enhancement of NK cell activity may be an under-lying mechanism of the anti-carcinogenic properties of Se. Thus, neoplasms that are NK cell sensitive could be prevented and/or responsive to Se therapy. Enqvist et. al in 2011 showed that selenite may be used to potentiate the anti-tumor cytotoxicity in settings of NKC-based immunotherapies. Hawkes et. al. in 2009 administered 42 men 300 μg of Se. Supplementation in-creased blood Se concentration by 50% and increased counts of NKC and T lymphocytes expressing both sub-units of the high affinity interleukin-2 receptor (IL₂R). Selenium supplementation can protect mice against the toxicity of iodine. Selenium, beta-carotene, and glutamine can also increase the number and/or cytotoxic activity of NKC. The antioxidant supplementation may be useful for the preservation of health and functional longevity in aging populations.

### Vitamin B Complex

Vitamin B (V_{B}) Complex is a compilation of eight different vitamins, known as vitamin B complex, found naturally in high-protein foods, such as fish, and leafy greens. Vitamin supplementation is associated with increased antibody titer response to both hepatitis B and tetanus vaccines as a result of macrophage and T cell stimulation. Because of these findings and others, nutraceuticals are becoming more widely accepted as an adjunct to conventional therapies for enhancing general well-being. High blood concentrations of folic acid (Vitamin B9), however, may be related to the decreased NK cell cytotoxicity, and high folate status may reduce the response to antifolate drugs used against malaria, rheumatoid arthritis, psoriasis, and cancer. According to Erkut et al, vitamin B12 may have important immunomodulatory effects on cellular immunity, and abnormalities in the immune system in pernicious anemia are restored by vitamin B12 replacement therapy. Vitamin B12 might play an important role in cellular immunity, especially relativing to CD8+ cells and the NK cell system, which suggests effects on cytotoxic cells. Tamura et al. concluded that Vitamin B12 acts as an immunomodulator for cellular immunity. The molecular mechanism responsible for the age-dependent decline of NK cell is restored.

It has been demonstrated that surgery may induce immunosuppression. This finding could influence the clinical course of surgically treated cancer patients and vitamin supplementation is warranted. Moreover, preliminary experimental studies have shown that a preoperative injection of IL-2 response is well known. IL-2-induced neutralization of postoperative lymphocytopenia is associated with a prolonged survival time in advanced colorectal cancer patients. Patients given 5-fluorouracil (5-FU)/IFN-alpha show augmentation of NK cell function and abrogation of 5-FU-induced suppression of LAK cell activity. While vitamin B6 status is not a primary etiological factor in HIV-1-related immunological dysregulation, it appears to be an important cofactor of immune function. It appears that vitamin B6 is an essential nutrient for maintenance of normal T-cell function in vivo.

### Vitamin C

Preserved function of the immune system is an excellent marker of health and longevity. Micronutrients such as zinc, selenium, iron, copper, beta-carotene, vitamins A, C, and E, and folic acid can influence several components of innate immunity. Deficiencies in zinc and vitamins A and D may reduce NK cell function, whereas supplemental zinc or Vitamin C (V_{C}) may enhance their activity. The improvement of leukocyte functions after ingestion of Vitamin C suggests that this antioxidant supplementation may be useful for the preservation of health and functional longevity in aging populations. In one study, the in vitro effects of Vitamin C and selenium on NK cell activity of ss-thalassemia major patients were investigated and found that Vitamin C enhanced NK cell activity significantly. As antioxidant diet supplementation decreases oxidative stress, it may be useful to treat hypertension and increase longevity in agreement with the oxidation/inflammation theory of aging. Combinations of 1 mM for thioproline and NAC and 5 μM for Vitamin C and vitamin E induced enhancement of the NK cell activity at all ages studied. This stimulation of the NK cell activity by antioxidants is an important favorable response, especially in old mice, in which age results in a decrease in NK cell function and, therefore, in a higher incidence of neoplasia.

After exposure to many toxic chemicals, NK cell function can be decreased significantly. Immune functional abnormalities can be restored after toxic chemical exposure by oral usage of Vitamin C. No convincing data exist that moderate exercise training is linked to improve T helper cell counts in patients with HIV, or enhance immunity in elderly participants. The plasma level of interleukin (IL)-6 and IL-1 receptor antagonist increased 20- and 3-fold after exercise. The plasma level of creatine kinase was increased six-fold the day after exercise. The concentrations of CD4+ memory T cells, CD8+ memory and naïve T cells, and NKC increased at the end of exercise. These effects may be related to the increase Nutraceuticals taken by active individuals, which include vitamins. Natural products such as Vitamin C may increase cytotoxic activity of NKC. Tumor Necrosis Factor alpha (TNF-alpha) decreases DNA damage in patients with late-stage cancer. A combination of Nutraceuticals can raise NK cell function and TNF-alpha activity and result in improved clinical out-comes in patients with late stage cancer. In a study of 20 patients with stage 1V, end-stage cancers were given vitamin C. As of a mean of 6 months, 16/20 patients were still alive. The 16 survivors had significantly higher NKC function than baseline (p<0.01 for each). Vitamin C has been found to enhance NK cell activity and antibody de-pendent cellular cytotoxicity (ADCC). This increased survival was completely abrogated when NK cell and killer (K) cell activities were depleted by cyclophosphamide. Platelets aggregation around migrating tumor cells offers protection against the cytotoxic activity of the NKC. Ascorbic acid inhibited platelet aggregation in-creased the NK cell cytotoxicity for 285%. The results suggest the role of ascorbic acid in increasing the susceptibility of tumor cells to NK cell; the Vitamin C could be used as part of a multidrug therapy to treat diseases which up to now have been treated only through chemotherapy. A study was reported to examine the effect of 500 to 5,000 mg of Vitamin C on DNA adducts on NK cell activity. At doses greater than 500 mg, this cellular absorption was not increased further, and all doses produced equivalent in-creases in ascorbic acid on days 1 to 15. After a period of 1 week, with no placebo or vitamin washout, Vitamin C levels along with functional assays returned to the baseline and became equivalent to placebos. These studies have concluded that Vitamin C was an antioxidant and that doses up to 5,000 mg did not induce mutagenic lesions nor had negative effects on NK cell activity, apoptosis, or the cell cycle. Vitamin C concentrations in the plasma and leukocytes rapidly de-cline during infections and stress. Supplementation of Vitamin C was found to improve components of the human immune system such as antimicrobial and NK cell activities. Zinc deficiency was been shown to also impair cellular mediators of innate immunity such as phagocytosis, NK cell activity, and the generation of oxidative burst. Therefore, both nutrients play important roles in immune function and the modulation of host resistance to infectious agents. A large number of randomized controlled intervention trials with intakes of up to 1 g of vitamin C and up to 30 mg of zinc are available. These trials document that adequate intakes of vitamin C and zinc ameliorate symptoms and shorten the duration of respiratory tract infections including the common cold.

### Vitamin D3

A novel inflammatory crosstalk between NKC and eosinophils via IL-15/IL-8 axis can be modulated by vitamin D3. Vitamin D3 increases the susceptibility of cancer cells to NKC and may therefore have a preventive role in melanoma occurrence or potentiate the anticancer effects of NK cell-cell immune therapy. The vitamin D3 up regulated protein 1 (VDUP1) and is a stress-response gene that is up regulated by 1,25-dihydroxyvitamin D3 (1,25(OH) 2D3) in tumor cells. Lee et al. suggested that VDUP1 is a critical factor for the development and function of NKC in vivo. Low vitamin D status is associated with an increased risk of immune-mediated diseases like inflammatory bowel disease (IBD) in humans. Acute myeloid leukemia (AML) displays low levels of ligands for the activating immune-receptor NKCG2D and will evade NK cell immune-surveillance. These findings identify NKCG2D ligands are thus targets of leukemia differentiation therapy and suggest a clinical benefit in combining a pharmacological approach with NKC-based immunotherapy in AML. Several lines of evidence suggest that 1,25-dihydroxyvitamin D3 (1,25 D3) may be important in chemoprevention of human cancer. Human promyelocytic leukemia cells HL60 cultured in the presence of 30 nM 1,25 D3 for three years exhibited a reduced rate of tumor growth when injected into nu/nu mice. These results suggest a mechanism for the reported chemopreventive effects of sunlight-generated 1,25 D3 or dietary vitamin D3. Given the importance of proper invariant NKCT (iNKCT) cell function in health and disease, this prenatal requirement for vitamin D suggests that in humans, the amount of vitamin D available in the environment during prenatal development may dictate the number of iNKCT cells and potential risk of autoimmunity.

### Vitamin E

Cancer patients with advanced disease display signs of immune suppression, which constitute a major obstacle for effective immunotherapy. Both T cells and NKC are affected by a multitude of mechanisms of which the generation of reactive oxygen species is of major importance. DCs based vaccines have only exhibited minimal effectiveness against established tumors in mice and humans. Tocotrienol-rich fraction (TRF) has the potential to be an adjuvant to augment DC based immunotherapy. Two weeks of high-dose treatment with the anti-oxidant vitamin E enhanced NK cell function in cancer patients by protecting from oxidative stress and in patients with colorectal cancer. The improvement of leukocyte functions after ingestion of thiolic antioxidants suggests that antioxidant supplementation may be useful for the preservation of health and functional longevity in aging populations. Immune response is impaired in the elderly but NK cell activity increased in supplemented subjects and decreased in non supplemented individuals. Inter-leukin-2 production by PBMC and the proportion of T cells with NK cell activity decreased in controls and did not change in supplemented subjects. Supplemented subjects reported less infection than no supplemented individuals (in 13% and 22% of scheduled visits, respectively; p=0.02). Supplements included nutrients in parts, 480 kcal, 31 g proteins, 120 IU vitamin E, 3.8 ug vitamin B12, 400 ug folic acid. The powerful influence of host nutritional status on the course of viral infection has been shown. The role of alpha-vitamin E and Se on human lymphocyte oxidative stress and T-cells proliferation indicated that alpha-vitamin E and Se have interactive effects as oxygen radical scavengers, thus promoting human lymphocyte response to antigens. Twenty male recreational runners randomly received either anti-oxidants (500 mg of vitamin C and 400 mg of vitamin E) or placebo for 14 days before and 7 days after a 5% downhill 90-min treadmill run at 75% VO (2 max). The plasma level of interleukin (IL)-6 and IL-1 receptor antagonist increased 20- and 3-fold after exercise. In the supplemented group, plasma level of creatine kinase was increased sixfold the day after exercise. The concentrations of CD4+ memory T cells, CD8+ memory and naïve T cells, and NK cell increased at the end of exercise. On the other hand exercise in a person who is unconditioned or unaccustomed to exercise will induce oxidative damage and resulted in muscle injury.

### Zinc

Zinc (Zn) is one of the most important trace elements in the body and it is essential as a catalytic, structural and regulatory ion. It is involved in homeostasis, in immune responses, in oxidative stress, in apoptosis and in aging. Deficiency of zinc is prevalent worldwide in developing countries and may affect nearly 2 billion subjects. The major manifestations of zinc deficiency include growth retardation, hypogonadism in males, cell-mediated immune dysfunctions, and cognitive impairment. Zinc not only improves cell mediated immune functions but also functions as an antioxidant and anti-inflammatory agent. Prasad et al. recommended further studies and propose that zinc should be utilized in the management and chemoprevention of cancer.

Zinc-binding proteins (metallothioneins, MTs), are protective in situations of stress and in situations of exposure to toxic metals, infections and low Zn nutrition. MTs play a key role in Zn-related cell homeostasis due to their high affinity for Zn, which is in turn relevant against oxidative stress and immune responses, including NK cell activity and aging, since NK cell activity and Zn ion bioavailability de-crease in aging. Physiological supplementation of Zn in aging and in age-related degenerative diseases corrects immune defects, reduces infection relapse and prevents aging. Zinc is not stored in the body and excess intakes result in reduced absorption and increased excretion. Nevertheless, there are cases of acute and chronic Zn poisoning. Aging is accompanied by chronic inflammation and oxidative stress, which lead to a marked impairment of immune function and therefore increased mortality. Data suggest that improvement of leukocyte function and restoration of redox balance after consumption of adequate levels of antioxidants from adulthood may be useful to attain healthy aging.

The promyelocytic leukemia zinc-finger (PLZF) is essential for the development of innate T cells (as represented by NKC T cells) for acquisition of their unique innate immune properties. The transcriptional regulator PLZF is highly expressed during the differentiation of NKC T (NKCT) cells and is essential for the acquisition of their effector/memory innate-like phenotype. The Senescence-Accelerated Mouse (SAM) represents a group of inbred mouse strains developed as a model for the study of human aging and age-related diseases. Schnurri (Shn)-2 is a large zinc finger-containing protein implicated in cell growth, signal transduction and lymphocyte development. In Shn-2(−/−) mice, Yamashita et al. observed decreased cytotoxicity of NKC accompanied by decreased expression of perforin and granzyme-B. Thus, Shn-2 is considered to play an important role in the activation and function of NKCs and the development of T cell lymphoma in vivo. Most liver NKCT cells failed to recirculate. Antibody blocking experiments established that they were retained locally through constitutive LFA-1-intercellular adhesion molecule (ICAM) 1 interactions. This unprecedented lifelong intravascular residence could be induced in conventional CD4 T cells by the sole expression of PLZF, a transcription factor specifically expressed in the NKCT lineage. The nutritional factor, zinc, may remodel these changes with subsequent healthy aging, because zinc improves the inflammatory/immune response as shown by in vitro and in vivo studies. Zinc supplementation in old C-carriers restores NK cell cytotoxicity and zinc status. The genetic variations of the IL-6-loci are useful tools for the choice of old people for zinc supplementation.

Interferons (IFNs) direct innate and acquired immune responses and, accordingly, are used therapeutically to treat a number of diseases. The promyelocytic leukemia zinc finger (PLZF) protein is part of the IFN response. During aging, dysregulated immune functions occur contributing to increased susceptibility to morbidity and mortality. NKC and NKC T (NKCT) cell cytotoxicity, representing one of the best models of innate immune response, decreases in aging as well as IFN-gamma production by both activated types of cells. The role played by zinc and MT is crucial because some MT polymorphisms are involved in maintaining innate immune response and intracellular zinc ion availability in aging with thus a role of MT genetic background to es-cape some age-related diseases with subsequent healthy aging and longevity. Muzzioli et al. evaluated the effect of zinc on the kinetic of development of CD34(+) stem cell progenitors towards NKC in young and old ages. CD34(+) cells was greatly lower in old than in young subjects. These cells progressively decreased in cultures from young subjects whereas they remained at very low levels in old donors. Zinc influences the proliferation and differentiation of CD34(+) progenitors both in young and old ages.

Down syndrome is associated with an increased susceptibility to infections due to the deficiency of both specific and nonspecific immunity. Plasma level of zinc is normal from birth until 5 years but with a temporal trend of progressive reduction. This observation supports the hypothesis that a pharmacological supplementation may be necessary in Down syndrome children only after 5 years of age. Pro-inflammatory cytokine response and NK cell activity are controlled by the availability of zinc ions, whose intra-cellular transport is regulated by angiogeneic metallothioneins. IL-6, MCP-1 and NK cell activities were evidenced following supplementation, indicating the genetic background as one of the determinants for identifying groups of subjects that can take advantage of therapeutic intervention. These results demonstrated an enhanced production of IL-12 during the acute phase of infection in zinc-supplied groups. So Brazão et al. concluded that zinc supplementation leads to an effective host's immune response by up-modulating the host's immune response, thus contributing to the reduction of blood parasites. Prasad et al. concluded that zinc has an important role in cell-mediated immune functions and also functions as anti-inflammatory and antioxidant agent.

The NKC maturation from CD34(+) hematopoietic cell precursors is a complex process that requires the synergistic effect of different cytokines and growth factors. Although there have been a number of important advances in the understanding of the NK cell differentiation, the developmental step leading to mature NK cell is still poorly defined. Zinc influences the proliferation and differentiation of CD34(+) progenitors. Nutritional intake of 10 mg of zinc increases the quantity of interferon-gamma-producing NK cell and strengthens the immune system against neoplasms and viral infections. It is very important to include assays of NK cell markers and functions in future longitudinal studies in order to investigate this point in detail as well as to consider the trace element zinc as an essential co-factor for optimal NK cell activity. Vitamin C concentrations in the plasma and leukocytes rapidly decline during infections and stress. Supplementation of vitamin C was found to improve components of the human immune system such as antimicrobial and NK cell activities, lymphocyte proliferation. Vitamin C and zinc reduce the incidence and improve the outcome of pneumonia, malaria, and diarrhea infections, especially in children in developing countries. Zinc prevents immune deficiencies in cell-mediated immunity in healthy 55-70 years old.

In reference to Table 1 below, it has summarized the mechanisms and disease targets for the supplements described above. In Table 1, Direct Anti-cancer Activity was shown by garlic, Lentinan, Resveratrol, Selenium, Vitamins C, D3, E and Zinc. Increased NK cell activity was shown by all but ALA and Vitamin B complex, which revealed mixed results in the studies. ALA and Vitamin B complex mainly acts as an anti-inflammatory and BRM. When supplements are used in cancer patients, those with both NK cell stimulatory and direct cytotoxicity functions to cancers would contribute to the demise of the cancer cells. Therefore, the mechanisms of killing, including the BRM, antiangiogenic, antioxidant and anti-inflammatory activities, would be difficult to determine in cancer patients. Determination of the actual NK cell role is compounded by some of the supplements being directly cytotoxic to cancer cells, which is actually beneficial to the patients.

TABLE 1
Summary of Mechanisms and Disease Targets for the Components:

| | NKC | Anti- | | |
|---|---|---|---|---|
| Component | Activity | Cancer | Mech. Action | Disease(s) |
| ALA¹ | ± | | Anti-inflammatory | Diabetes, MS |
| | | | | Alzheimer's |
| Arabinoxylan | + | | BRM², Apop³ | Cancer, |
| | | | | Respiratory |
| Curcumin | + | | Chemoprev⁴, Antiox⁵ | Cancer |
| | | | Antibacterial, Apop | |
| Garlic | + | + | Antiox, Apop | Cancer |
| Geinstein | + | | Antiag⁶ | Cancer |
| Ginseng | + | | | Cancer, MS |
| Lentinan | + | + | Erythropoiesis | Cancer |
| | | | | Anemia, Heart |
| Mistletoe | + | | | AIDS, Cancer |
| NAC | + | | Apop, Antiag | AIDS, Cancer |
| Resveratrol | + | + | BRM, Apop | Cancer, MDR |
| | | | | Longevity |
| Selenium | + | + | BRM | Cancer, |
| | | | | Longevity |
| Vitamin B | ± | | BRM | Arthritis, |
| | | | | Energy, Malaria |
| Vitamin C | + | + | Antiox | Longevity |
| | | | | Cancer, HIV |
| | | | | Exercise |
| Vitamin D₃ | + | + | Anti-inflammatory | Stess, chemo- |
| | | | BRM | prevention |
| Vitamin E | + | + | DC⁷, Antiox | Autoimmunity |
| Zinc | + | + | Anti-inflammatory | Cancer, |
| | | | Antiox | Antiaging, |
| | | | | Stem Cells |
| | | | | Down |
| | | | | Syndrome |

¹Alpha Lipoic Acid
²Biological Response Modifier
³Apoptosis
⁴Chemoprevention
⁵Antioxidant
⁶Anti-angiogenesis
⁷Dendritic Cells

Furthermore, in reference to Table 2 as set forth below, it reveals the occurrences of the synergism between the components of the formulation, which affect the viability of cancer cells. Synergy or synergism is the interaction of multiple elements in a system to produce an effect greater than the sum of their individual effects. While a true measure of the NK cell effect is complicated by synergistic and possibly even antagonist effects.

TABLE 2
The Synergism between Various Components:

| | |
|---|---|
| Arabinoxylan-Curcumin | [30] |
| Resveratrol-Vitamin C | [216] |
| NAC-Vitamin C | [287] |
| Vitamin C-Zinc | [256] |
| Vitamin E, B₁₂ Folic acid | [265] |
| Vitamin E-Selenium | [267] |

In light of the above disclosures, NK cell is shown to be pivotal in the body's process of recognizing and destroying cancer cells. And this is also true for other human or animal diseases, such as viral infections. There are no available data from clinical trials for NK cell therapy. However, in vivo animal and in vitro human NK cell killing of the tumor cells in co-cultures have indicated excellent and astonishingly positive effects in comparison with other treatments. Furthermore, empirical although sporadic and not systematically studied cases have shown that elevated NK cell counts found in some AIDS patients correlate with either long survival or very delayed outburst of the disease symptoms. The development of a combination of the supplements in the proper combinations into a tablet or capsule form would be an important part of the prevention and treatment of cancer that complements the classical approaches. Moreover, the formulation can be administered orally, subcutaneous, intravenously, as a suppository or aerosol spray that boosts the immune system of either human or animal. The very act of taking supplements with the education and encouragement of one's physician would go a long way in the health of the patient. Besides, physicians can encourage health and wellness to promote good mental health in their patients (and themselves), which leads to healthy life style changes, including eating the right foods, exercise, and adequate sleep with mental and spiritual well-being. The physician can greatly influence decreasing depression that follows a cancer diagnosis with a positive attitude that “one is doing something” and has the endorsement of the physician and family.

Certain studies have indicated that cancer is a malfunction of the immune system, and the NK cell is pivotal in preventing cancer from developing on a cellular level. To protect against cancer and treat it, a variety of treatments are necessary. In the same vein the prevention and treatment of cancer using supplements relies on a multifaceted approach. Therefore multiple components are necessary. The Formulation contains 16 necessary components. The Formulation addition of a “NKC tablet” will require advertising restricted to health and wellness, while advocating “not to be used for the diagnosis and treatment of any diseases”. The physicians along with the public will have to educate themselves from publications and the internet as to the value of the supplements. This educational process must be driven by the masses, since companies are restricted by the Food and Drug Administration. Since over 40% of cancer patients are taking supplements, and over 70% of the older public is taking some form of supplementation, the medical community has a duty to educate future physicians in the value of supplementation.

Although the invention has been explained in relation to its preferred embodiment, it is to be understood that many other possible modifications and variations can be made without departing from the spirit and scope of the invention as herein described.

### LIST OF ABBREVIATIONS

1,25(OH) 2D₃ 1,25-dihydroxyvitamin D3
1,25 D₃ 1,25-dihydroxyvitamin D3
4NQO 4-nitroquinoline 1-oxide
5-FU 5-fluorouracil
ABGE aged black garlic extracts
AD Alzheimer's disease
ADCC antibody-dependent cell-mediated cytotoxicity
AGE Aged garlic extract
ALA Alpha-lipoic acid
AmB Amphotericin B
AML Acute myeloid leukemia
APG Acidic Polysaccharide of Ginseng
ATL Adult T-cell leukemia
ATRA Chemotherapy for promyelocytic leukemia
AXOS Arabinoxylan-oligosaccharides
BAFs breast adipose fibroblasts
BioBran Arabinoxylan rice bran
BCC Breast cancer cell
BGP Boiled garlic powder
BRM biological response modifier
CAM Complementary and alternative medicine
cAMP cyclic AMP
CBDCA carboplatin
CD3+ T cell Marker
CD4+ Helper Cell Marker
CD8+Suppressor Cell Marker
CHAX corn husk arabinoxylan
CIPFC Complement Independent Plaque Forming Cell
CLL chronic lymphocyte leukemia
CPB cardiopulmonary bypass
CRC colorectal cancer
CY cyclophosphamide
DADS Diallyl disulfide
DAS Diallyl sulfide
DATS diallyl trisulfide
DCs dendritic cells
DMH 1,2-dimethylhydrazine
EC effector cells
EMT epithelial-mesenchymal transition
Epo erythropoietin
ER-α estrogen receptor
GEN Genistein
GSH glutathione
GSSG Glutathione disulfide
H2S Hydrogen sulfide
HepG2 human hepatoma
HL60 Human promyelocytic leukemia cells
HRB Hydrolyzed Rice Bran
IBD inflammatory bowel disease
ICAM intercellular adhesion molecule
IFN-alpha interferon-alpha
IFN-gamma interferon-gamma
IFNs Interferons
IL interleukin
IL-1 Interleukin-1
IL-2 Interleukin-2
IL-4 Interleukin-4
IL-6 Interleukin-6
IL-8 Interleukin-8
IL-12 Interleukin-12
IL-13 Interleukin-13
IL2R interleukin-2 receptor
iNKCT invariant NKCT
LA Lipoic acid
LAC lactacystin
LAK lymphokine activated killer
LDCC lectin-dependent cell-mediated cytotoxicity
LEN Lentinan
LGL granular lymphocytes
LPS Lipopolysaccharides
MAPK Mitogen Activated Protein Kinase
MCF-7 sensitive human breast carcinoma cells
MDR multidrug resistance
MEFs Murine embryonic fibroblasts
MGN-3 Arabinoxylan rice bran
MLs Mistletoe lectins
MM multiple myeloma
MMP-2 matrix metalloproteinase-2
MMPs matrix metalloproteinases
MPS myofascial pain syndrome
MRP2 multidrug resistance protein 2
MS multiple sclerosis
MTs metallothioneins
MW molecular weight
NASH non-alcoholic steatohepatitis
NFAT Nuclear Factor of Activated T-cells
NKC Natural Killer Cell
OSCC oral squamous cell carcinoma
OSCs organosulfur compounds
PAM prematurely aging mice
PBL peripheral blood lymphocytes
PBMC peripheral blood mononuclear cells
PC prostate cancer
PgR progesterone receptor
PKA Protein Kinase A
PFC Plaque Forming Cell
PLZF promyelocytic leukemia zinc-finger
PNAC peripheral non-adherent cells
PTK protein tyrosine kinase
PVR poliovirus receptor
RES Resveratrol
rIL-2 recombinant interleukin-2
ROS reactive oxygen species
RFC Rosette Forming Cells (T-cells)
SAC S-allyl cysteine
SAM Senescence-Accelerated Mouse
SAMC S-allylmercaptocysteine
SEM Scanning Electron Microscopy
SCD1 stearoyl-CoA desaturase-1
SCT stem cell transplantation
Se Selenium
SFN Sulforaphane
SMI Shenmai injection
SRBC Sheep Red Blood Cells
TNF-α tumor necrosis factor-alpha
TP thioproline
TEM Transmission Electron Microscopy
TRF Tocotrienol-rich fraction
Vcr-R resistant human breast carcinoma cells
VDUP1 vitamin D3 up regulated protein 1
VEGF Vascular Endothelial Growth Factor
XIAP X-linked inhibitor of apoptosis
Zn Zinc

What is claimed is:

1. A supplement formulation for preventing and treating human and animal diseases, comprising
said supplement formulation boosting natural killer cells (NKC) in human or animal body;
said supplement formulation comprising multiple components; and
said supplement formulation comprising synergy between said multiple components.

2. The supplement formulation for preventing and treating human and animal diseases as set forth in claim 1, comprising
said supplement formulation boosting the natural killer cells (NKC) by increasing quantity of the natural killer cells (NKC) in a human or animal body; and
said supplement formulation boosting the natural killer cells (NKC) by enhancing activity of the natural killer cells (NKC) in a human or animal body.

3. The supplement formulation for preventing and treating human and animal diseases as set forth in claim 1, comprising
arabinoxylan;
curcumin; and
the arabinoxylan and the curcumin having the synergy.

4. The supplement formulation for preventing and treating human and animal diseases as set forth in claim 1, comprising
resveratrol;
Vitamin C; and
the resveratrol and the Vitamin C having the synergy.

5. The supplement formulation for preventing and treating human and animal diseases as set forth in claim 1, comprising
N-acetylcysteine;
Vitamin C; and
the N-acetylcysteine and the Vitamin C having the synergy.

6. The supplement formulation for preventing and treating human and animal diseases as set forth in claim 1, comprising
Zinc;
Vitamin C; and
the Zinc and the Vitamin C having the synergy.

7. The supplement formulation for preventing and treating human and animal diseases as set forth in claim 1, comprising
Vitamin E;
Vitamin B Complex; and
the Vitamin E and the Vitamin B Complex having the synergy.

8. The supplement formulation for preventing and treating human and animal diseases as set forth in claim 1, comprising
Vitamin E;
Selenium; and
the Vitamin E and the Selenium having the synergy.

9. The supplement formulation for preventing and treating human and animal diseases as set forth in claim 1, comprising
Alpha lipoic acid (ALA), Arabinoxylin, Curcumin, Garlic, Genistein, Ginseng, Lentinan, Mistletoe, N-Acetylcysteine, Resveratrol, Selenium, and Zinc.

10. The supplement formulation for preventing and treating human and animal diseases as set forth in claim 1, comprising
Alpha lipoic acid (ALA), Arabinoxylin, Curcumin, Garlic, Genistein, Ginseng, Lentinan, Mistletoe, N-Acetylcysteine, Resveratrol, Selenium, Vitamin B Complex, Vitamin C, Vitamin D₃, and Vitamin E.

11. The supplement formulation for preventing and treating human and animal diseases as set forth in claim 1, comprising
Arabinoxylin, Curcumin, Garlic, Genistein, Ginseng, Lentinan, Mistletoe, N-Acetylcysteine, Resveratrol, Selenium, Vitamin B Complex, Vitamin C, Vitamin D₃, Vitamin E and Zinc.

12. The supplement formulation for preventing and treating human and animal diseases as set forth in claim 1, comprising
Alpha lipoic acid (ALA), Curcumin, Garlic, Genistein, Ginseng, Lentinan, Mistletoe, N-Acetylcysteine, Resveratrol, Selenium, Vitamin B Complex, Vitamin C, Vitamin D₃, and Vitamin E.

13. The supplement formulation for preventing and treating human and animal diseases as set forth in claim 1, comprising
Alpha lipoic acid (ALA), Arabinoxylin, Garlic, Genistein, Ginseng, Lentinan, Mistletoe, N-Acetylcysteine, Resveratrol, Selenium, Vitamin B Complex, Vitamin C, Vitamin D₃, and Vitamin E.

14. The supplement formulation for preventing and treating human and animal diseases as set forth in claim 1, comprising
Alpha lipoic acid (ALA), Arabinoxylin, Genistein, Lentinan, Mistletoe, N-Acetylcysteine, Resveratrol, Selenium, Vitamin B Complex, Vitamin C, Vitamin D₃, and Vitamin E.

15. The supplement formulation for preventing and treating human and animal diseases as set forth in claim 1, comprising
Alpha lipoic acid (ALA), Arabinoxylin, Garlic, Genistein, Ginseng, Lentinan, N-Acetylcysteine, Resveratrol, Selenium, Vitamin B Complex, Vitamin C, Vitamin D₃, and Vitamin E.

16. The supplement formulation for preventing and treating human and animal diseases as set forth in claim 1, comprising
Alpha lipoic acid (ALA), Arabinoxylin, Garlic, Ginseng, Lentinan, Mistletoe, N-Acetylcysteine, Resveratrol, Selenium, Vitamin B Complex, Vitamin C, Vitamin D₃, and Vitamin E.

17. The supplement formulation for preventing and treating human and animal diseases as set forth in claim 1, comprising
Alpha lipoic acid (ALA), Arabinoxylin, Garlic, Genistein, Ginseng, Lentinan, Mistletoe, N-Acetylcysteine, Vitamin B Complex, Vitamin C, Vitamin D₃, and Vitamin E.

18. The supplement formulation for preventing and treating human and animal diseases as set forth in claim 1, comprising
Alpha lipoic acid (ALA), Arabinoxylin, Curcumin, Garlic, Genistein, Ginseng, Lentinan, Mistletoe, N-Acetylcysteine, Resveratrol, Selenium, Vitamin B Complex, Vitamin C, Vitamin D₃, Vitamin E and Zinc.

19. The supplement formulation for preventing and treating human and animal diseases as set forth in claim 1, comprising
the formulation comprising forms of tablet, capsule suppository and aerosol spray; and
the formulation being administered orally, subcutaneous, trans-dermal, or intravenously.

20. The supplement formulation for preventing and treating human and animal diseases as set forth in claim 1, wherein
the human and animal diseases comprising cancer and viral infection.
